(19) United States
(12) Patent Application Publication
(10) Pub. No.: US 2023/0159652 A1
(43) Pub. Date: May 25, 2023

(54) TRANSFERRIN RECEPTOR 1 TARGETING FOR CARCINOGENESIS PREVENTION

(71) Applicant: The Regents of the University of California, Oakland, CA (US)

(72) Inventors: Manuel L. Penichet, Los Angeles, CA (US); Otoniel M. Martinez, Los Angeles, CA (US); Marta Epeldegui, Los Angeles, CA (US); Tracy R. Wells, Los Angeles, CA (US); Laura E. Martinez, Los Angeles, CA (US)

(73) Assignee: The Regents of the University of California, Oakland, CA (US)

(21) Appl. No.: 17/907,036

(22) PCT Filed: Mar. 23, 2021
(86) PCT No.: PCT/US2021/023735

§ 371 Date: Sep. 22, 2022

Related U.S. Application Data
(60) Provisional application No. 62/993,530, filed on Mar. 23, 2020.

(51) Int. Cl. C07K 16/28; A61K 39/395; A61K 47/68; A61K 45/06; A61P 35/00; A61P 31/22

(52) U.S. Cl. CPC C07K 16/2881; A61K 39/3955; A61K 47/6877; A61K 45/06; A61P 35/00; A61P 31/22; C07K 2317/24; C07K 2317/21; A61K 2039/505

(57) ABSTRACT

Aspects of the disclosure relate to transferrin receptor 1 (TfR1) binding proteins and methods of use. In some cases, methods and compositions for preventing cancer comprising the use of TfR1-binding proteins are described. Embodiments include methods for preventing cancer, for example cancer caused by an infectious agent (e.g., Epstein-Barr virus), using a TfR1-binding protein. In some embodiments, the disclosed methods and compositions involve one or more antibodies that are capable of binding TfR1. Certain aspects relate to chimeric antibodies and antibody-like molecules.

### CROSS REFERENCE TO RELATED APPLICATIONS

This application claims the benefit of priority of U.S. Provisional Patent Application No. 62/993,530 filed Mar. 23, 2020, which is hereby incorporated by reference in its entirety.

### BACKGROUND

This invention was made with government support under Grant Numbers CA196266 and CA228157, awarded by the National Institutes of Health. The government has certain rights in the invention.

### Field of the Invention

This invention relates generally to the fields of molecular biology, immunology, immunotherapy, and medicine.

### Background

Transferrin receptor 1 (TfR1), or CD71, is a cell surface protein responsible for facilitating iron uptake into cells via binding to iron-loaded transferrin protein. TfR1 expression is increased on a wide variety of cancer cells, including hematopoietic cancers. In some cases, such as in chronic lymphocytic leukemia (CLL) and non-Hodgkin lymphoma (NHL), its expression can be correlated with tumor stage or prognosis. Additionally, TfR1 is expressed on endothelial cells of the blood brain barrier (BBB), and is used by certain viruses for cell entry. See Daniels et al., Clin. Immunol., 121(2):144-158 (2006); Daniels et al., Biochim. Biophys. Acta., 1820(3):291-317 (2012); Daniels-Wells et al., Immunotherapy, 8(9):991-994 (2016); and Helguera et al., J. Virol., 86(7):4024-4028 (2012).

Antibodies targeting TfR1 may provide various benefits in treating conditions such as cancer and viral infections. Chimeric antibodies targeting TfR1 have been developed (see, e.g., Daniels et al., J. Immunother., 34(6):500-508 (2011); Helguera et al., J. Virol., 86(7):4024-4028 (2012); and Daniels-Wells et al., J. Immunother., 43(2):48-52 (2020), each of which are incorporated herein by reference in their entirety). Mouse/human chimeric IgG3/kappa and IgG1/kappa antibodies targeting TfR1 have been developed for therapeutic uses including, for example, as cancer therapies (see, e.g., Daniels-Wells et al., Toxicol. In Vitro, 27(1):220-231 (2013); Daniels-Wells et al., J. Immunother., 38(8):307-310 (2015); Leoh et al., J. Gene Med., 16(1-2):11-27 (2014); Leoh et al., J. Immunol., 200(10):3485-3494 (2018); and Sun et al., Cancer Res., 79(6):1239-1251 (2019), each of which are incorporated herein by reference in their entirety). These chimeric antibodies (ch128.1/IgG3 and ch128.1/IgG1) contain the variable regions of the murine monoclonal antibody 128.1 specific for human TfR1 (Daniels et al., J. Immunother., 34(6):500-508 (2011) and Daniels-Wells et al., J. Immunother., 43(2):48-52 (2020), incorporated herein by reference).

Epstein-Barr virus (EBV) is a human γ-herpesvirus that is nearly ubiquitous in human populations, with over 90% of adults, worldwide, infected with EBV (Farrell, Annu. Rev. Path., 14:29-53 (2019); Tosato and Blaese, Adv. Immunol., 37:99-149 (1985)). EBV is a B-cell-tropic virus that has significant oncogenic potential. Infection with EBV is life-long, and after initial infection, which sometimes causes infectious mononucleosis, is asymptomatic in healthy, immunocompetent persons. However, ongoing control of EBV infection requires a vigorous, life-long immune response. Therefore, EBV-associated cancers are a particular problem in immunodeficient persons, such as those infected with the human immunodeficiency virus (HIV) or organ-transplant recipients, who are immunosuppressed to avoid transplant rejection. (Shannon-Lowe et al., Philosophical Transactions of the Royal Society of London, Series B, Biological Sciences, 372, 20160271 (2017); Marques-Piubelli et al., Pathology 52: 40-52 (2020)). For example, EBV infection is associated with several hematopoietic cancers of B-cell origin, including acquired immunodeficiency syndrome (AIDS)-related non-Hodgkin lymphomas (NHL) and Hodgkin lymphoma (HL), as well as African/endemic Burkitt lymphoma, primary effusion lymphoma (PEL), and EBV⁺ B-cell lymphoma of the elderly, who demonstrate an age-related decrease in adaptive immunity (Tsao, Philosophical Transactions of the Royal Society of London. Series B, Biological Sciences, 372, 20160270 (2017)). Additionally, EBV infection of B-cells is seen in post-transplant lymphoproliferative disorders (PTLD), which is characterized by polyclonal B-cell hyperplasia, which can give rise to lymphoma (Martinez, Pediatr. Nephrol., 35(7):1173-1181(2020)). EBV is also associated with non-hematopoietic cancers, including nasopharyngeal carcinoma and gastric cancer (Nishikawa et al., Cancers, 10(6): Article 167 (2018); Young and Dawson, Chin. J. Cancer, 33(12):581-590 (2014); Tsao et al., J. Pathol., 235(2):323-333 (2015)). Initial infection of B-cells with EBV results in polyclonal B-cell activation, driving cellular proliferation and immunoglobulin (Ig) production (Martinez-Maza and Britton, J. Exp. Med., 157(6):1808-1814 (1983); Rosen et al., Nature, 267(5606):52-54 (1977); Yarchoan, J. Exp. Med. 157(1):1-14 (1983)). Some days after initial infection, B-cells infected by EBV undergo transformation and immortalization, leading to EBV⁺ clones that grow indefinitely, in the absence of effective immune system control. The emergence of transformed, immortalized B-lymphoblastoid cells lines (BLCL) occurs 6-8 weeks following initial EBV infection in vitro, characterized by the outgrowth of one or more dominant clones, with a marked increase in cell number and cellular proliferation. The hematopoietic cancers that are associated with EBV infection result from ineffective immune system control of EBV-infected B-cells, allowing these cells to undergo activation and transformation, and then to evolve into lymphoma (Crombie and LaCasce, Front. Oncol., 7: Article 109 (2019)). Therefore, a therapeutic intervention that can effectively prevent EBV-driven B-cell activation and/or transformation is needed and has the potential to prevent the development of B-cell hematopoietic cancers, such as AIDS-NHL or PTLD-associated lymphomas.

### SUMMARY

The methods and compositions disclosed herein are based, at least in part, on the discovery that TfR1-binding proteins such as chimeric anti-TfR1 antibodies are capable of inhibiting carcinogenesis. Certain aspects are directed to prevention of carcinogenesis by elimination of pre-malignant cells using TfR1-binding proteins. Particular embodiments encompass the use of TfR1-binding proteins in the prevention of a cancer caused by and/or associated with an infectious agent. For example, disclosed herein are methods for preventing development of EBV-driven B-cell hematopoietic cancers using anti-TfR1 antibodies.

Embodiments of the present disclosure include, inter alia, methods and compositions for preventing cancer comprising TfR1-binding proteins. Antigen-binding proteins described herein may be used in preventing one or more types of cancer associated with a TfR1 protein such as, for example, B-cell lymphoma. In some embodiments, antigen-binding proteins are used to prevent cancer, for example cancer caused by or associated with an infectious agent.

Embodiments include compositions comprising one or more antigen-binding proteins (e.g., TfR1-binding proteins). Embodiments include humanized antibodies or antibody-like molecules. Embodiments also include nucleic acid molecules encoding for one or more antigen-binding proteins or portions thereof. Embodiments include recombinant, transformed or modified cells, vectors, and/or expression cassettes comprising such nucleic acid molecules. In some embodiments, the compositions contemplated herein can comprise 1, 2, 3, 4, 5, or more of the following components: an antigen-binding protein, a nucleic acid, a vector, a cell, a polypeptide, an oligonucleotide, a light chain variable region, a heavy chain variable region, a light chain constant region, and a heavy chain constant region. Any one or more of these components may be excluded from the disclosed compositions.

Embodiments also include methods of generating an antigen-binding protein, methods of producing an antigen-binding protein, methods of expressing an antigen-binding protein, methods of humanizing a chimeric antigen-binding protein, methods of detecting TfR1, methods of treating one or more conditions, methods of purifying TfR1, methods of treating cancer, methods of preventing cancer, methods of inhibiting carcinogenesis, and methods of eliminating one or more cells expressing TfR1. The steps and embodiments discussed in this disclosure are contemplated as part of any of these methods. In some embodiments, the methods contemplated herein can comprise or exclude 1, 2, 3, 4, 5, or more of the following steps: providing an antigen-binding protein, detecting an infectious agent, providing a nucleic acid to a cell, subjecting a cell to conditions sufficient to express a nucleic acid, providing an additional therapeutic, covalently attaching a therapeutic to an antigen-binding protein, non-covalently attaching a therapeutic to an antigen-binding protein, expressing a vector in a cell, and providing a pharmaceutical composition to a subject. Any one or more of these steps may be excluded from the disclosed methods.

In some embodiments, the antigen-binding protein has or lacks one or more post-translational modifications such as myristoylation, palmitoylation, isoprenylation or prenylation, farnesylation, geranylgeranylation, glypiation, acylation, acetylation, formylation, alkylation, methylation, amide bond formation, amidation at C-terminus, arginylation, polyglutamylation, polyglycylation, butyrylation, glycosylation, glycation, polysialylation, malonylation, hydroxylation, iodination, phosphorylation, adenylylation, propionylation, S-glutathionylation, S-nitrosylation, S-sulfenylation (aka S-sulphenylation), succinylation, sulfation, biotinylation, PEGylation, SUMOylation, ubiquitination, neddylation, pupylation, disulfide bridges, or racemization. In other embodiments, the antigen-binding protein has reduced or increased amounts of one or more post-translational modifications as compared to the same antigen-binding protein expressed in the cell that is native to the encoded gene. The reduction or increase may be by at least or at most 25, 50, 60, 70, 80, 90, 100, 150, 200, 250, 300, 350, 400, 450, 500% or more (or any range derivable therein). In some embodiments, the TfR1-binding protein has a binding affinity for TfR1 of between 0.001 and 1000 nM. In some embodiments, the TfR1-binding protein has a binding affinity for TfR1 of between 0.01 and 100 nM. In some embodiments, the TfR1-binding protein has a binding affinity for a TfR1 protein of between 0.1 and 20 nM. In some embodiments, the TfR1-binding protein has a binding affinity for a TfR1 protein of between 1 and 10 nM. In some embodiments, the TfR1-binding protein has a binding affinity for a TfR1 protein of at most, at least, or about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 20.5, 21, 21.5, 22, 22.5, 23, 23.5, 24, 24.5, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, or 1000 nM, or any range derivable therein.

In some embodiments, the TfR1-binding protein is an antibody, an antibody-like molecule, or a fragment thereof. In some embodiments, the TfR1-binding protein is an antibody, a nanobody, a minibody, an scFv fragment, or an Fab fragment. In some embodiments, the TfR1-binding protein is an antibody. In some embodiments, the TfR1-binding protein is a chimeric antibody. In some embodiments, the TfR1-binding protein is a ch128.1 antibody. In some embodiments, the TfR1-binding protein is ch128.1/IgG1. In some embodiments, the TfR1-binding protein is ch128.1/IgG3.

Certain aspects are directed to a composition comprising a TfR1-binding protein, such as a TfR1-binding protein described herein, and an additional therapeutic. In some embodiments, the additional therapeutic is covalently attached to the TfR1-binding protein. In some embodiments, the additional therapeutic is non-covalently attached to the TfR1-binding protein. In some embodiments, the additional therapeutic is a chemotherapeutic drug, a nucleic acid (e.g., an antisense oligonucleotide, a small interfering RNA (siRNA), or a clustered regularly interspaced short palindromic repeats (CRISPR)-based gene therapy), a protein (e.g., a toxin or an enzyme), a viral vector, or a nanodrug.

Other embodiments are directed to a use of the composition comprising the TfR1-binding protein, which in some embodiments is linked to a therapeutic agent, in the manufacture of a medicament for the prevention of cancer. Certain embodiments are directed to use of the composition comprising the TfR1-binding protein in the manufacture of a medicament for the prevention of cancer. In some embodiments, the TfR1-binding protein is used in the manufacture of a medicament for the prevention of cancer caused by or associated with an infectious agent.

Aspects of the disclosure are directed to methods of preventing cancer development or progression. In some embodiments, the disclosed methods comprise preventing, reversing, or slowing carcinogenesis in a subject. In some embodiments, disclosed is a method for preventing cancer comprising providing to a subject a TfR1-binding protein. In some embodiments, the cancer is a cancer caused by and/or associated with an infectious agent. In some embodiments, disclosed is a method for preventing cancer comprising a) detecting the presence of an infectious agent in the subject; and b) providing to the subject an effective amount of a TfR1-binding protein. In some embodiments, the infectious agent is EBV. In some embodiments, the infectious agent is hepatitis B virus (HBV), hepatitis C virus (HCV), Epstein-Barr virus (EBV), human papillomavirus (HPV), human immunodeficiency virus (HIV), human T-cell leukemia/lymphoma virus type 1 (HTLV-1), Kaposi sarcoma-associated herpesvirus (KSHV), Merkel cell polyomavirus (MCPyV), or HIV (e.g., HIV-1). In some embodiments, the cancer is non-Hodgkin lymphomas (NHL), Hodgkin lymphoma (HL), Burkitt lymphoma, primary effusion lymphoma (PEL), B-cell lymphoma of the elderly, adult T-cell leukemia/lymphoma (ATL), Kaposi sarcoma, hepatocellular carcinoma, cervical cancer, gastric cancer, or nasopharyngeal carcinoma. In some embodiments, the cancer is NHL. In some embodiments, the cancer is HL. In some embodiments, the subject has or is at risk for developing post-transplant lymphoproliferative disorder (PTLD). In some embodiments, the method further comprises providing an additional therapeutic. In some embodiments, the additional therapeutic is covalently or non-covalently attached to the TfR1-binding protein. In some embodiments, the additional therapeutic is not attached to the TfR1-binding protein. In some embodiments, the additional therapeutic is a chemotherapeutic, a toxin, an antisense oligonucleotide, a small inhibitory RNA (siRNA), an enzyme, a viral vector, a protein, or a nanodrug. In some embodiments, preventing or slowing carcinogenesis comprises eliminating pre-malignant cells from a subject. In some embodiments, preventing, reversing, or slowing carcinogenesis comprises inhibiting proliferation of pre-malignant cells from a subject.

Particular embodiments of the present disclosure are directed to a method for preventing EBV-associated cancer comprising: (a) detecting the presence of EBV in a subject; and (b) providing to the subject an effective amount of ch128.1/IgG1.

Further embodiments of the disclosure are directed to a method for preventing cancer comprising providing to a subject an effective amount of a TfR1-binding protein. In some embodiments, the method further comprises detecting an infectious agent in the subject. Detecting an infectious agent in a subject may comprise, for example, direct detection of the agent (e.g., via PCR-based detection, antibody-based detection, etc.) or indirect detection of the agent (e.g., via detection of downstream effects of the agent). In some embodiments, the infectious agent is detected prior to providing the TfR1-binding protein to the subject. In some embodiments, the infectious agent is detected subsequent to providing the TfR1-binding protein to the subject. In some embodiments, the TfR1-binding protein is a ch128.1 antibody. In some embodiments, the ch128.1 antibody is ch128.1/IgG1.

It is contemplated that any method or composition described herein can be implemented with respect to any other method or composition described herein and that different embodiments may be combined.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more compositions may be employed based on any of the methods described herein. Other embodiments are discussed throughout this application. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Example section are understood to be embodiments that are applicable to all aspects of the technology described herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a timeline of the treatment of mice in the experiments described in Example 1.

FIG. 2 shows results from the in vivo efficacy study described in Example 1, demonstrating that treatment with ch128.1/IgG1 enhances survival in mice implanted with EBV-infected B -cell enriched preparations.

FIGS. 3A-3D show the location and appearance of tumor-like lesions in the organs of representative mice implanted with EBV⁺ B-cell enriched preparations treated with the isotype (IgG1) control antibody (not treated with ch128.1/IgG1). FIG. 3D shows normal (no tumor-like growths noted) gross tissue images from a representative mouse implanted with EBV-exposed B-cells and treated with ch128.1/IgG1 antibody.

FIG. 4 shows a representative flow cytometry analysis of human cells that grew in mice implanted with EBV⁺ B-cell enriched preparations, demonstrating the effect of treatment with ch128.1/IgG1, which greatly reduced the growth of human cells when compared to controls.

FIGS. 5A-5B show the effect of ch128.1/IgG treatment in reducing the fraction of human B-cells, determined by flow cytometry, in different organs in mice implanted with EBV⁺ B-cell enriched preparations.

FIG. 6 shows immunohistochemical analysis of tumor-like growths that developed in mice implanted with EBV⁺ B-cell enriched preparations and treated with isotype control antibody, confirming that these tumor-like lesions were of human B-cell origin (CD19⁺), EBV⁺ (LMP1⁺) and clonal (either human kappa (κ) or lambda (λ)).

FIGS. 7A-7H show plasma cytokine, chemokine, and soluble receptor levels, determined using a multiplexed fluorescent bead-based immunometric assay, from mice treated as described in Example 1.

FIGS. 8A-8B show plasma immunoglobulin (Ig) free light chain (FLC) κ and λ levels, determined by enzyme-linked immunosorbent assay (ELISA), from mice treated as described in Example 1.

FIGS. 9A-9F show plasma immunoglobulin isotype (IgA, IgG1, IgG2, IgG3, IgG4, and IgM) levels, determined by multiplexed immunometric assay, from mice treated as described in Example 1.

### DETAILED DESCRIPTION

As disclosed herein in some aspects, TfR1 targeting, e.g., providing a TfR1-binding protein such as the antibody ch128.1/IgG1, is useful in the treatment and prevention of cancers associated with an infectious agent, such as virally-associated cancer. Accordingly, aspects of the present disclosure are directed to prevention of the development or progression of an EBV-associated cancer (e.g., lymphoma) by administering an effective amount of an anti-TfR1 antibody, such as ch128.1/IgG1 or ch128.1/IgG3, to a subject having or at risk for developing the EBV-associated cancer. Certain aspects pertain to methods of preventing carcinogenesis using an anti-TfR1 antibody. Also disclosed are compositions for use in such treatment methods.

### I. Definitions

“Individual, “subject,” and “patient” are used interchangeably and can refer to a human or non-human.

The terms “lower,” “lowered,” “reduce,” “reduced,” “reduction,” “decrease,” “decreased,” “inhibit,” “inhibited,” or “inhibition” are all used herein generally to mean a decrease by a statistically significant amount. However, for avoidance of doubt, “lower,” “lowered,” “reduce,” “reduced,” “reduction,” “decrease,” “decreased,” “inhibit,” “inhibited,” or “inhibition” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

The terms “increased,” “increase,” “enhanced,” “enhance,” “activated,” or “activate” are all used herein to generally mean an increase by a statistically significant amount; for the avoidance of any doubt, “increased,” “increase,” “enhanced,” “enhance,” “activated,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ end of the DNA/RNA corresponding to the (amino) terminus (N-terminus) of the protein and a translation stop codon at the 3′ end of the DNA/RNA whose prior codon corresponds to the (carboxy) terminus (C-terminus) of the protein. A gene can include, but is not limited to, cDNA from prokaryotic or eukaryotic mRNA, genomic DNA sequences from prokaryotic or eukaryotic DNA, and synthetic DNA sequences. A transcription termination sequence will be located 3′ to the gene sequence.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive “or”.

Throughout this application, the term “about” is used to indicate that a value includes the inherent variation of error for the measurement or quantitation method.

### II. Antibodies

Aspects of the disclosure relate to antibodies that specifically bind to TfR1. In some embodiments, the disclosed antibodies are mouse, chimeric, or humanized anti-TfR1 antibodies. In some embodiments, the disclosed antibodies are chimeric anti-TfR1 antibodies.

The term “antibody” refers to an intact immunoglobulin of any class or isotype, or a fragment thereof that can compete with the intact antibody for specific binding to the target antigen, and includes chimeric, humanized, and fully human antibodies. Also contemplated are antibodies having specificity for more than one antigen or target, including bispecific antibodies, trispecific antibodies, tetraspecific antibodies, and other multispecific antibodies. As used herein, the terms “antibody” or “immunoglobulin” are used interchangeably and refer to any of several classes of structurally related proteins that function as part of the immune response of an animal, including IgM, IgD, IgG, IgA, IgE, and related proteins, as well as polypeptides comprising antibody complementarity-determining regions (CDRs) that retain antigen-binding activity. Antibodies from various species are contemplated, including but not limited to human, mouse, goat, horse, rabbit, donkey, bovine, canine, chicken, feline, guinea pig, hamster, monkey, rat, sheep, and pig antibodies.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable of binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen would recognize an epitope on the target antigen within a complex mixture.

The epitope regions of a given polypeptide can be identified using many different epitope mapping techniques well known in the art, including: x-ray crystallography, nuclear magnetic resonance spectroscopy, site-directed mutagenesis mapping, protein display arrays, and hydrogen-deuterium exchange see, e.g., Rockberg and Nilvebrant (Eds.), Epitope Mapping Protocols, Humana Press, New York, N.Y., USA (2018). Such techniques are known in the art and described in, e.g., U.S. Pat. No. 4,708,871; Geysen et al., Proc. Natl. Acad. Sci., USA 81(13):3998-4002 (1984); Geysen et al., Proc. Natl. Acad. Sci., USA 82:178-182 (1985); and Geysen et al., Mol. Immunol., 23(7):709-715 (1986), each of which is incorporated by reference herein in their entirety. Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, for chimeric antibodies, the variable regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front. Immunol., 4: Article 302 (2013)).

The term “light chain” may describe a full-length light chain or fragments thereof. A full-length light chain has a molecular weight of around 25,000 Daltons and includes a variable region domain (abbreviated herein as V_{L}), and a constant region domain (abbreviated herein as CL). There are two classifications of light chains, identified as kappa (κ) and lambda (λ). The term “V_{L} fragment” means a fragment of the light chain of a monoclonal antibody that includes all or part of the light chain variable region, including CDRs. A V_{L} fragment can further include light chain constant region sequences. The variable region domain of the light chain is at the amino-terminus of the polypeptide.

The term “heavy chain” may describe a full-length heavy chain or fragments thereof. For example, a full-length heavy chain for human IgG1 has a molecular weight of around 50,000 Daltons and includes a variable region domain (abbreviated herein as V_{H}), and three constant region domains (abbreviated herein as C_{H}1, C_{H}2, and C_{H}3). The term “V_{H} fragment” means a fragment of the heavy chain of a monoclonal antibody that includes all or part of the heavy chain variable region, including CDRs. A V_{H} fragment can further include heavy chain constant region sequences. The number of heavy chain constant region domains will depend on the isotype. The isotype of an antibody can be IgM, IgD, IgG, IgA, or IgE and is defined by the heavy chains present of which there are five classifications: mu (μ), delta (d), gamma (γ), alpha (α), or epsilon (ε) chains, respectively. Human IgG has several subtypes, including, IgG1, IgG2, IgG3, and IgG4.

### A. Types of Antibodies

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one immunoglobulin unit. Monomers are the basic functional units of antibodies. The term “dimer” means an antibody containing two immunoglobulin units attached to one another via constant domains of the antibody heavy chains (the Fc, or fragment crystallizable, region). The complex may be stabilized by a joining (J) chain protein. The term “multimer” means an antibody containing more than two immunoglobulin units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificity or they may be bispecific, meaning the two antigen-binding sites have different antigen specificities.

Bispecific antibodies are a class of antibodies that have paratopes (i.e., antigen-binding sites) for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies may be sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86(15):7875-7882 (2014), each of which are specifically incorporated herein by reference in their entirety.

Bispecific antibodies can be constructed as: a whole IgG, Fab′2, Fab′PEG, a diabody, or alternatively as a single chain variable fragment (scFv). Diabodies and scFvs can be constructed without an Fc region, using only variable domains. Bispecific antibodies may be produced by a variety of methods including, but not limited to, fusion of hybridomas or linking of Fab′ fragments. See, e.g., Songsivilai and Lachmann, Clin. Exp. Immunol. 79(3):315-321 (1990); Kostelny et al., J. Immunol. 148(5):1547-1553 (1992), each of which are specifically incorporated by reference in their entirety.

In certain aspects, the antigen-binding domain may be multispecific or heterospecific by multimerizing with V_{H} and V_{L} region pairs that bind a different antigen. Accordingly, aspects may include, but are not limited to, bispecific, trispecific, tetraspecific, and other multispecific antibodies or antigen-binding fragments thereof that are directed to epitopes and to other targets, such as Fc receptors on effector cells. The antibody may bind to, or interact with, (a) a cell surface antigen, (b) an Fc receptor on the surface of an effector cell, or (c) at least one other component.

In some embodiments, multispecific antibodies can be used and directly linked via a short flexible polypeptide chain, using routine methods known in the art. One such example is diabodies that are bivalent, bispecific antibodies in which the V_{H} and V_{L} domains are expressed on a single polypeptide chain, and utilize a linker that is too short to allow for pairing between domains on the same chain, thereby forcing the domains to pair with complementary domains of another chain creating two antigen-binding sites. The linker functionality is applicable for embodiments of triabodies, tetrabodies, and higher order antibody multimers (see, e.g., Hollinger et al., Proc. Natl. Acad. Sci., USA, 90(14):6444-6448 (1993); Polijak et al., Structure, 2(12):1121-1123 (1994); and Todorovska et al., J. Immunol. Methods, 248(1-2):47-66 (2001), each of which is incorporated herein by reference in their entirety).

The part of the Fv fragment of an antibody molecule that binds with high specificity to the epitope of the antigen is referred to herein as the “paratope.” The paratope consists of the amino acid residues that contact the epitope of an antigen to facilitate antigen recognition. Each of the two Fv fragments of an antibody is composed of the two variable domains, V_{H} and V_{L}, in dimerized configuration. The primary structure of each of the variable domains includes three hypervariable loops separated by, and flanked by, framework regions (FRs). The hypervariable loops are the regions of highest primary sequences variability among the antibody molecules from any mammal. The term hypervariable loop is sometimes used interchangeably with the term “complementarity determining region (CDR).” The length of the hypervariable loops (or CDRs) varies between antibody molecules. The FRs of all antibody molecules from a given mammal have high primary sequence similarity/consensus. The consensus of FRs from different antiboides—typically from the same species—can be used by one skilled in the art to identify both the FRs and the hypervariable loops (or CDRs) which are interspersed among the FRs. The hypervariable loops are given identifying names which distinguish their position within the polypeptide, and on which domain they occur. CDRs in the V_{L} domain are identified as L1 (also CDR-L1), L2 (also CDR-L2), and L3 (also CDR-L3), with L1 occurring at the most distal end with respect to the C_{L} domain and L3 occurring closest to the C_{L} domain. The CDRs may also be given the names CDR1, CDR2, and CDR3. The L3 (CDR3) is generally the region of highest variability in the V_{L} domain among all antibody molecules produced by a given organism. The CDRs are regions of the polypeptide chain arranged linearly in the primary structure and separated from each other by FRs. The amino terminal (N-terminal) end of the V_{L} chain is named FR1. The region identified as FR2 occurs between L1 and L2 hypervariable loops. FR3 occurs between L2 and L3 hypervariable loops, and the FR4 region is closest to the C_{L} domain. This structure and nomenclature are repeated for the V_{H} chain, which includes three CDRs identified as H1 (also CDR-H1), H2 (also CDR-H2), and H3 (also CDR-H3). The H3 (CDR-H3) is generally the region of highest variability in the antibody molecules produced by a given organism. The majority of amino acid residues in the variable domains, or Fv fragments (V_{H} and V_{L}), are part of the FRs (approximately 85%).

Several methods have been developed and can be used by one skilled in the art to identify the amino acids that constitute each of these regions. This can be done using any of a number of multiple sequence alignment methods and algorithms, which identify the conserved amino acid residues that make up the FRs, therefore identifying the CDRs that may vary in length but are located between FRs. Three commonly used numberings have been developed for identification of the CDRs of antibodies: Kabat (as described in Wu and Kabat, J. Exp. Med., 132(2): 211-250 (1970)); Chothia (as described in Chothia et al., Nature, 342(6252): 877-883 (1989)); and IMGT (as described in Lefranc et al., Dev. Comp. Immunol., 27(1): 55-77 (2003)). These methods each include unique numbering systems for the identification of the amino acid residues that constitute the variable regions. In most antibody molecules, the amino acid residues that actually contact the epitope of the antigen occur in the CDRs, although in some cases, residues within the FRs contribute to antigen-binding. Depending on the type and size of the antigen, different CDR residues may contact the antigen. See Almagro, J. Mol. Recognit., 17(2):132-43 (2004), incorporated herein by reference.

One skilled in the art can use any of several methods to determine the paratope of an antibody. These methods include:

1) Computational predictions of the tertiary structure of the antibody/epitope binding interactions based on the chemical nature of the amino acid sequence of the antibody variable region and composition of the epitope.

2) Hydrogen-deuterium exchange and mass spectroscopy.

3) Polypeptide fragmentation and peptide mapping approaches in which one generates multiple overlapping peptide fragments from the full length of the polypeptide and evaluates the binding affinity of these peptides for the epitope.

4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific V_{H} and V_{L} domains as appropriate.

5) X-ray crystallography.

6) Alanine scanning mutagenesis.

In certain aspects, affinity matured antibodies are enhanced with one or more modifications in one or more CDRs thereof (and/or one or more FRs thereof) that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Biotechnology, 10(7):779-783 (1992) describes affinity maturation by V_{H} and V_{L} domain shuffling, random mutagenesis of CDR and/or FRs employed in phage display is described by Rajpal et al., Proc. Natl. Acad. Sci. USA, 102(24): 8466-8471 (2005) and Thie et al., Methods Mol Biol., 525:309-322 (2009) in conjugation with computation methods as demonstrated in Tiller et al., Front. Immunol., 8: Article 986 (2017), each of which references are incorporated herein by reference in their entirety.

Chimeric immunoglobulins are the products of fused genes derived from different species; “humanized” antibodies generally have the FRs from human immunoglobulins and one or more CDRs are from a non-human source (e.g., murine).

In some embodiments, minimizing the antibody polypeptide sequence from the non-human species optimizes chimeric antibody function and reduces immunogenicity. Specific amino acid residues of the non-human antibody are modified to be homologous to corresponding residues in a human antibody. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. In some instances, corresponding non-human (e.g., murine) residues replace FR amino acid residues of the human immunoglobulin. Replacement of human FR residues with non-human FR residues may serve to improve and/or restore antigen-binding. Furthermore, humanized antibodies may comprise residues that are not found in the recipient antibody or in the donor antibody to further refine performance. The humanized antibody may also comprise at least a portion of an immunoglobulin constant region, typically that of a human immunoglobulin.

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

A monoclonal antibody or “mAb” refers to an antibody obtained from a population of homogeneous antibodies from an exclusive parental cell, e.g., the population is identical except for naturally occurring mutations that may be present in minor amounts. Each monoclonal antibody is directed against a single antigenic determinant (epitope).

### B. Functional Antibody Fragments and Antigen-Binding Fragments

### 1. Antigen-Binding Fragments

Certain aspects relate to antibody fragments, such as antibody fragments that bind to antigen. The term functional antibody fragment includes antigen-binding fragments of an antibody that retain the ability to specifically bind to an antigen. These fragments are constituted of various arrangements of the variable region heavy chain (V_{H}) and/or light chain (V_{L}); and in some embodiments, include constant region heavy chain 1 (C_{H}1) and light chain (C_{L}). In some embodiments, they lack the Fc region constituted of heavy chain 2 (C_{H}2) and 3 (C_{H}3) domains. Embodiments of antigen-binding fragments and the modifications thereof may include: (i) the Fab fragment type constituted with the V_{L}, V_{H}, C_{L}, and C_{H}1 domains; (ii) the Fd fragment type constituted with the V_{H} and C_{H}1 domains; (iii) the Fv fragment type constituted with the V_{H} and V_{L} domains; (iv) the single domain fragment type, dAb, (Holt et al., Trends Biotechnol., 21(11):484-490 (2003)) constituted with a single V_{H} or V_{L} domain; (v) isolated CDRs. Such terms are described, for example, in Harlow and Lane (Eds.), Antibodies: A Laboratory Manual, Cold Spring Harbor Laboratory, New York, N.Y., USA (1988); Molecular Biology and Biotechnology: A Comprehensive Desk Reference, Meyers (Ed.), Wiley-VCH Publisher, Inc., New York, N.Y., USA (1995); Huston et al., Cell Biophys., 22(1-3):189-224 (1993); and Pluckthun and Skerra, Methods Enzymol., 178:497-515 (1989), each of which are incorporated by reference in their entirety.

Antigen-binding fragments also include fragments of an antibody that retain exactly, at least, or at most 1, 2, or 3 CDRs from a light chain variable region. Fusions of CDR-containing sequences to an Fc region (or a C_{H}2 or C_{H}3 region thereof) are included within the scope of this definition including, for example, scFv fused, directly or indirectly, to an Fc region are included herein.

The term Fab fragment means a monovalent antigen-binding fragment of an antibody containing the variable (V_{L} and V_{H}) and the constant (C_{L} and C_{H}1) domains. The term Fab′ fragment means a monovalent antigen-binding fragment of a monoclonal antibody that is larger than a Fab fragment. For example, a Fab′ fragment includes the V_{L}, V_{H}, C_{L} and C_{H}1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two V_{H} and V_{L} domains and can further include all or part of the two C_{L} and C_{H}1 domains.

The term Fd fragment means a fragment of the heavy chain of a monoclonal antibody, which includes all or part of the V_{H}, including the CDRs. An Fd fragment can further include C_{H}1 region sequences.

The term Fv fragment means a monovalent antigen-binding fragment of a monoclonal antibody, including all or part of the V_{L} and V_{H}, and absent of the C_{L} and C_{H}1 domains. The V_{L} and V_{H} include, for example, the CDRs. Single-chain antibodies (sFv or scFv) are Fv molecules in which the V_{L} and V_{H} regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)₂ means bivalent or bispecific sFv polypeptide chains that include oligomerization domains at their C-termini, separated from the sFv by a hinge region. The oligomerization domain comprises self-associating α-helices, e.g., leucine zippers, which can be further stabilized by additional disulfide bonds. (scFv)₂ fragments are also known as “miniantibodies” or “minibodies.”

A single domain antibody is an antigen-binding fragment containing only a V_{H} or the V_{L} domain. In some instances, two or more V_{H} regions are covalently joined with a peptide linker to create a bivalent domain antibody. The two V_{H} regions of a bivalent domain antibody may target the same or different antigens.

### 2. Fragment Crystallizable (Fc) Region

In some cases, including for IgG, IgD, and IgA antibodies, an Fc region contains two heavy chain fragments comprising the C_{H}2 and C_{H}3 domains of an antibody. The two heavy chain fragments are held together by two or more disulfide bonds and by hydrophobic interactions of the C_{H}3 domains. The term “Fc polypeptide” as used herein includes native and mutein forms of polypeptides derived from the Fc region of an antibody. Truncated forms of such polypeptides containing a hinge region that promotes dimerization are included.

### 3. Cell Surface Receptors

Antigen-binding proteins of the present disclosure may be expressed on the surface of a cell. In some embodiments, antigen-binding proteins are cell surface receptors comprising antigen-binding domains (e.g., TfR1-binding domains) disclosed herein. In some embodiments, described herein are cell surface receptors comprising a TfR1-binding domain and one or more additional components or domains. Examples of cell surface receptors of the present disclosure include chimeric antigen receptors (CARs). A TfR1-specific cell surface receptor may comprise one or more of an antigen-binding domain, a signal peptide, an extracellular spacer, a transmembrane domain, a cytoplasmic region, and a linker. Cells expressing a TfR1-specific cell surface receptor may be useful in preventing one or more TfR1 associated conditions, as described elsewhere herein.

### C. Polypeptides with Antibody CDRs and Scaffolding Domains that Display the CDRs

Antigen-binding peptide scaffolds, such as CDRs, are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify (Skerra, J. Mol. Recognit., 13(4):167-187 (2000)).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibitors of neuronal nitric oxide synthase (PIN) may also be used.

### D. Antibody Binding

The term “selective binding agent”, “antigen-binding agent”, or “antigen-binding protein” refers to a molecule that binds to an antigen. Non-limiting examples include antibodies, antigen-binding fragments, scFv, Fab, Fab′, F(ab′)₂, single chain antibodies, aptamers, peptides, peptide fragments, and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (K_{D} or Kd), using the equation K_{D}=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the K_{D} are mol/L (molarity, or M), or concentration. The Ka of an antibody is the inverse of the K_{D}, and is determined by the equation Ka=1/K_{D}. Examples of some experimental methods that can be used to determine the K_{D} value are: enzyme-linked immunosorbent assays (ELISA), isothermal titration calorimetry (ITC), fluorescence anisotropy, surface plasmon resonance (SPR), and affinity capillary electrophoresis (ACE).

Antibodies deemed useful in certain embodiments may have an equilibrium dissociation constant of about, at least about or at most about 10⁻⁶, 10⁻⁷, 10⁻⁸, 10⁻⁹, 10⁻¹⁰ M, 10⁻¹¹ M, 10⁻¹² M, or any range derivable therein.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B- and/or T-cell receptors respond or recognize. Polypeptide epitopes can be formed from both contiguous amino acids and noncontiguous amino acids juxtaposed by tertiary folding of a polypeptide. In some embodiments, an epitope includes at least 3, for example 5-10 amino acids, in a unique spatial conformation.

Epitope specificity of an antibody can be determined in a variety of ways. One approach, for example, involves testing a collection of overlapping peptides of about 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N- and C-terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antigen-Binding Domains

It is understood that the antibodies of the present disclosure may be modified, such that they are substantially identical to the antibody polypeptide sequences, or fragments thereof, and still bind the epitopes of the present disclosure. Polypeptide sequences are “substantially identical” when optimally aligned using such programs as Clustal Omega, IGBLAST, GAP, or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present disclosure, providing that the variations in the amino acid sequence maintain at least 75%, more preferably at least 80%, at least 90%, at least 95%, at least 96%, at least 97%, at least 98% and most preferably at least 99% sequence identity. In some embodiments, conservative amino acid replacements are contemplated.

Conservative replacements (also “conservative substitutions” or “conservative amino acid substitutions”) are those that take place within a family of amino acids that possess similar biochemical properties, including charge, hydrophobicity, and size. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). Thus, a conservative replacement may comprise replacement of an amino acid in one family for an amino acid in the same family (e.g., replacement of a lysine with an arginine, replacement of an aspartate for a glutamate, etc.). Alternatively or in addition, amino acid similarity may be determined using a Blocks Substitution Matrix (BLOSUM), such as BLOSUM62 (Henikoff and Henikoff, Proc. Natl. Acad. Sci. USA, 89(22): 10915-9 (1992)). In this case, a conservative replacement may be a substitution of amino acids having a non-negative value on a BLOSUM62 matrix. Whether an amino acid change results in a functional peptide can readily be determined by assaying the specific activity of the polypeptide derivative. Standard ELISA, SPR, or other antibody-binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Certain preferred N- and C-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art (Dill and MacCallum, Science, 338(6110):1042-1046 (2012)). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

It is also contemplated that the antigen-binding domain may be multi-specific or multivalent by multimerizing the antigen-binding domain with V_{H} and V_{L} region pairs that bind either the same antigen (multi-valent) or a different antigen (multi-specific).

### E. Enzymatic or Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861, incorporated herein by reference). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be PEGgylated to increase the biological half-life by reacting the polypeptide with polyethylene glycol (PEG) or a reactive ester or aldehyde derivative of PEG, under conditions in which one or more PEG groups become attached to the polypeptide. Polypeptide PEGylation may be carried out by an acylation reaction or an alkylation reaction with a reactive PEG molecule (or an analogous reactive water-soluble polymer). Methods for PEGylating proteins are known in the art and can be applied to the polypeptides of the disclosure to obtain PEGylated derivatives of antibodies. See, e.g., EP 0154316 and EP 0401384, incorporated herein by reference. In some aspects, the antibody is conjugated or otherwise linked to transthyretin (TTR) or a TTR variant. The TTR or TTR variant can be chemically modified with, for example, a chemical selected from the group consisting of dextran, poly(n-vinylpyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen-binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin/streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses). In some embodiments, an antibody or fragment thereof is covalently attached to a molecule or substance, such as a labeling moiety or a therapeutic moiety. In some embodiments, an antibody or fragment thereof is non-covalently attached to a molecule or substance, such as a labeling moiety or a therapeutic moiety.

Optionally, an antibody or an antigen-binding fragment can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0486525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Example conjugated molecules are a chemotherapeutic drug, a nucleic acid (e.g. an antisense oligonucleotide, a siRNA or a CRISPR-based gene therapy, etc.), a protein (e.g. a toxin, an enzyme, etc.), a viral vector, or a nanodrug. Additional suitable conjugated molecules include ribonuclease (RNase), DNase I, an antisense oligonucleotide, an inhibitory RNA molecule such as a siRNA molecule, an immunostimulatory nucleic acid, aptamers, ribozymes, triplex forming molecules, and external guide sequences (e.g., guide RNAs). The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

### a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., avidin and streptavidin) and the like. Particular examples of labels are, but not limited to, fluorescein, fluorescein isothiocyanate (FITC), rhodamine, dansyl, umbelliferone, dimethyl acridinium ester (DMAE), Texas red, phycoerythrin (PE), and luminol. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase (AP), horseradish peroxidase (HRP), α- or β-galactosidase, and glucose oxidase. Preferred secondary binding ligands are avidin and streptavidin compounds that are capable of binding biotin with high affinity. The uses of such labels is well known to those of skill in the art and are described, for example, in U.S. Pat. Nos. 3,817,837; 3,850,752; 3,939,350; 3,996,345; 4,277,437; 4,275,149; and 4,366,241, each incorporated herein by reference. Molecules containing azido groups may also be used to form covalent bonds to proteins through reactive nitrene intermediates that are generated by low intensity ultraviolet light.

In some aspects, contemplated are immunoconjugates comprising an antibody or antigen-binding fragment thereof conjugated (e.g., covalently attached) to a cytotoxic agent such as a chemotherapeutic agent, a drug, a nucleic acid (e.g., antisense oligonucleotide, siRNA, etc.) a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing the targeted cell surface antigen. The antibody and the agent may be associated through non-covalent interactions such as through electrostatic forces, or by covalent bonds. Various linkers, known in the art, can be employed in order to form the immunoconjugate. Additionally, the immunoconjugate can be provided in the form of a genetic fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADCs), an antibody is conjugated to one or more drug moieties (e.g., small molecule drugs such as chemotherapeutics) through a linker. The ADCs may be prepared by several routes, employing organic chemistry reactions, conditions, and reagents known to those skilled in the art, including: (1) reaction of a nucleophilic group of an antibody with a bivalent linker reagent, to form antibody-L, via a covalent bond, followed by reaction with a drug moiety D; and (2) reaction of a nucleophilic group of a drug moiety with a bivalent linker reagent, to form drug-linker (D-L), via a covalent bond, followed by reaction with the nucleophilic group of an antibody. ADCs may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In certain aspects, ADCs include covalent or aggregative conjugates of antibodies, or antigen-binding fragments thereof, with other proteins or peptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo., USA) peptide as described in Hopp et al., Bio/Technology, 6:1204-1210 (1988) and U.S. Pat. No. 5,011,912.

Also contemplated herein are activatable immunoconjugates comprising an antibody or antigen binding fragment thereof conjugated to a therapeutic agent, and further comprising a masking moiety, wherein the masking moiety reduces the ability of the antibody or antigen-binding fragment thereof to bind to an antigen (e.g., TfR1). A masking moiety may be conjugated to an antigen-binding protein of the disclosure via a linker having a protease cleavage site, where the masking moiety is removed via protease activity in a tumor microenvironment, thereby activating the antigen-binding protein. Certain non-limiting examples of activatable antibodies, antibody fragments, and immunoconjugates (e.g., ADCs) are described in U.S. Pat. No. 10,179,817, incorporated herein by reference. In some embodiments, disclosed is an activatable anti-TfR1 antibody or antigen-binding fragment thereof.

### b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., J. Immunol. Methods, 99(2):153-161 (1987)).

### F. Proteins

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed; however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

Nucleotide as well as protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide per ml. The concentration of polypeptide in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### 1. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines its functional activity, certain amino acid substitutions can be made in a protein sequence and in its corresponding DNA coding sequence, and nevertheless produce a protein with similar or desirable properties.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ nucleic acid sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual amino acid residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative” (also “nonconservative”) In some embodiments, a non-conservative substitution affects a function or activity of the polypeptide. In some embodiments, a non-conservative substitution does not affect a function or activity of the polypeptide. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### G. Nucleic Acids

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, polymerase chain reaction (PCR) primers or sequencing primers for identifying, analyzing, mutating, or amplifying a polynucleotide encoding a polypeptide, antisense oligonucleotides for inhibiting expression of a polynucleotide, and complementary sequences of the foregoing described herein. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, 99%, or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, or at least 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000, or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### 1. Mutation

Changes can be introduced by mutation into a nucleic acid, thereby leading to changes in the amino acid sequence of a polypeptide (e.g., an antibody or antibody derivative) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449(3):581-594 (2013), incorporated herein by reference. For example, the mutation can quantitatively or qualitatively change the biological activity. Examples of quantitative changes include increasing, reducing, or eliminating the activity. Examples of qualitative changes include altering the antigen specificity of an antibody.

### III. Antibody Production

### A. Full-length Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745, each incorporated herein by reference (see, e.g., Harlow and Lane (Eds.), Antibodies: A Laboratory Manual, Cold Spring Harbor Laboratory, New York, N.Y., USA (1988); incorporated herein by reference). These antibodies may be polyclonal or monoclonal antibody preparations, monospecific antisera, human antibodies, chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques.

In an example, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography.

Methods of making monoclonal antibodies are also well known in the art (e.g., U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide, or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after about 2-3 weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA, 93:7843-7848 (1996)), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as high-performance liquid chromatography (HPLC). Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, interleukin-1 (IL-1), IL-2, IL-4, IL-7, IL-12, interferon-γ (INF-γ), granulocyte-macrophage colony-stimulating factor (GM-CSF), Bacillus Calmette-Guérin (BCG), aluminum hydroxide, muramyl dipeptide (MDP) compounds, muramyl tripeptide phosphatidyl ethanolamine (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, cytokines such as IFN-β, IL-2, or IL-12, or genes encoding proteins involved in immune helper functions, such as B7-1 (CD80) or B7-2 (CD86). A phage-display system can be used to expand antibody molecule populations in vitro.

### B. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv (see, e.g., Inbar et al., Proc. Nat. Acad. Sci. USA, 69(9):2659-2662 (1972); Hochman et al., Biochem., 15(12):2706-2710 (1976); and Ehrlich et al., Biochem., 19(17):4091-4096 (1980)).

Single-chain variable fragments (scFvs) may be prepared by fusing DNA encoding a peptide linker between DNA molecules encoding the two variable domain polypeptides (V_{L} and V_{H}). scFvs can form antigen-binding monomers, or they can form multimers (e.g., dimers, trimers, or tetramers), depending on the length of a flexible linker between the two variable domains. By combining different V_{L}- and V_{H}-comprising polypeptides, one can form multimeric scFvs that bind to different epitopes. Antigen-binding fragments are typically produced by recombinant DNA methods known to those skilled in the art. Although the two domains of the Fv fragment, V_{L} and V_{H}, are coded for by separate genes, they can be joined using recombinant methods by a synthetic linker that enables them to be made as a single chain polypeptide (known as single chain Fv (sFv or scFv); see e.g., Bird et al., Science, 242(4877):423-426 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by N-terminal and/or C-terminal deletions, where the remaining amino acid sequence is substantially identical to the corresponding positions in the naturally occurring sequence deduced, for example, from a full-length cDNA sequence.

Also contemplated herein are non-peptide compounds having properties analogous to those of a template peptide. These types of non-peptide compounds are termed “peptide mimetics” or “peptidomimetics”.

Also contemplated are “antibody like binding peptidomimetics” (ABiPs), which are peptide-like molecules that act as pared-down antibodies and have certain advantages of longer serum half-life as well as less cumbersome synthesis methods. These analogs can be peptides, non-peptides or combinations of peptide and non-peptide regions. Peptide mimetics that are structurally similar to therapeutically useful peptides may be used to produce a similar therapeutic or prophylactic effect. Such compounds are often developed with the aid of computerized molecular modeling. Generally, peptidomimetics of the disclosure are proteins that are structurally similar to an antibody displaying a desired biological activity, such as the ability to bind a protein, but have one or more peptide linkages optionally replaced by a linkage selected from: —CH2NH—, —CH2S—, —CH2-CH2-, —CH—CH— (cis and trans), —COCH2-, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain embodiments of the disclosure to generate more stable proteins. In addition, constrained peptides comprising a consensus sequence or a substantially identical consensus sequence variation may be generated by methods known in the art (Rizo and Gierasch, Ann. Rev. Biochem., 61:387-418 (1992), incorporated herein by reference), for example, by adding internal cysteine residues capable of forming intramolecular disulfide bridges which cyclize the peptide.

Once generated, a phage display library can be used to improve the immunological binding affinity of Fab molecules using known techniques (see, e.g., Figini et al., J. Mol. Biol., 239(1):68-78 (1994)). The coding sequences for the heavy and light chain portions of the Fab molecules selected from the phage display library can be isolated or synthesized and cloned into any suitable vector or replicon for expression. Any suitable expression system can be used.

### IV. Obtaining Antibodies

In some aspects, there are nucleic acid molecules encoding antibody or antibody-like polypeptides (e.g., heavy or light chain, variable domain only, or full-length). These may be generated by methods known in the art, e.g., isolated from B-cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, antigen-binding fragments, immunoadhesins, diabodies, bispecific antibodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and/or probes thereof. In addition to control sequences that govern transcription and translation, vectors and expression vectors may contain nucleic acid sequences that serve other functions as well.

To express the antibodies, or antigen-binding fragments thereof, DNA encoding partial or full-length light and heavy chains are inserted into expression vectors such that the gene area is operatively linked to transcriptional and translational control sequences. In some aspects, a vector that encodes a functionally complete human C_{H} or C_{L} immunoglobulin sequence with appropriate restriction sites engineered so that any V_{H} or V_{L} sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include, but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624; 5,981,274; 5,945,100; 5,780,448; 5,736,524; 5,702,932; 5,656,610; 5,589,466; and 5,580,859, each incorporated herein by reference), including microinjection (U.S. Pat. No. 5,789,215, incorporated herein by reference); by electroporation (U.S. Pat. No. 5,384,253, incorporated herein by reference); by calcium phosphate precipitation; by using DEAE dextran followed by polyethylene glycol; by direct sonic loading; by liposome mediated transfection; by microprojectile bombardment (PCT Publication Nos. WO 94/09699 and 95/06128; U.S. Pat. Nos. 5,610,042; 5,322,783; 5,563,055; 5,550,318; 5,538,877; and 5,538,880, and each incorporated herein by reference); by agitation with silicon carbide fibers U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); or by PEG mediated transformation of protoplasts (U.S. Pat. Nos. 4,684,611 and 4,952,500, each incorporated herein by reference); by desiccation/inhibition mediated DNA uptake. Other methods include viral transduction, such as gene transfer by lentiviral or retroviral transduction.

### 4. Host Cells

In another aspect, contemplated are the use of host cells into which a recombinant expression vector has been introduced. Antibodies and antibody-like molecules can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to immune cell (e.g., T-cell) activation. Control of antibody expression allows immune cells, such as tumor-targeting immune cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors and their cognate polypeptides. Host cells which may be used to express antibodies and other antigen-binding proteins of the present disclosure include, for example, murine myeloma cells (e.g., NS0/1 cells, SP2/0-Ag14 cells, and P3X63Ag8.653 cells), Chinese hamster ovary (CHO) cells, baby hamster kidney 21 (BHK21) cells, human embryonic kidney 293 cells (HEK293), fibrosarcoma cells (HT-1080), and the human embryonic retinal cells PER.C6.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. The sequences of human heavy and light chain constant region genes are also known in the art. Nucleic acid molecules encoding the full-length heavy and/or light chains may then be expressed in a cell into which they have been introduced and the antibody isolated.

### V. Methods of Treatment and Administration of Therapeutic Compositions

The therapy provided herein may comprise administration of a therapeutic agent (e.g., a TfR1-binding protein). In some embodiments, therapy provided herein comprises administration of a combination of therapeutic agents, such as a TfR1-binding protein and an additional therapeutic agent. An additional therapeutic may be an additional cancer therapeutic. An additional therapeutic may be a chemotherapy. The therapy or therapies may be administered in any suitable manner known in the art. For example, for a combination therapy, the TfR1-binding protein and the additional therapeutic agent may be administered sequentially (at different times) or concurrently (at the same time). In some embodiments, the TfR1-binding protein and the additional therapeutic agent are administered in a separate composition. In some embodiments, the TfR1-binding protein and the additional therapeutic agent are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The various therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or μM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

### A. General Pharmaceutical Compositions

In some embodiments, pharmaceutical compositions are administered to a subject. Different aspects may involve administering an effective amount of a composition to a subject. In some embodiments, an antibody or antigen-binding fragment capable of binding to TfR1 is administered to the subject to protect against or treat a condition (e.g., cancer). Alternatively, an expression vector encoding one or more such antibodies or polypeptides or peptides may be given to a subject as a preventative treatment. Additionally, such compositions can be administered in combination with an additional therapeutic agent (e.g., a chemotherapeutic, immunotherapeutic). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

### B. Methods of Treatment

Compositions (e.g., antigen-binding proteins) or methods described herein may be administered to any patient at risk for developing a condition in which targeting TfR1 may have therapeutic benefit. Conditions in which targeting TfR1 may have a therapeutic benefit include, for example, a condition associated with the expression of TfR1 (e.g., cancer).

The term “treatment” or “treating” means any treatment of a disease in a mammal, including:

(i) preventing the disease, that is, causing the clinical symptoms of the disease not to develop by administration of a protective composition prior to the induction of the disease;

(ii) suppressing the disease, that is, causing the clinical symptoms of the disease not to develop by administration of a protective composition after the inductive event but prior to the clinical appearance or reappearance of the disease;

(iii) inhibiting the disease, that is, arresting the development of clinical symptoms by administration of a protective composition after their initial appearance; and/or

(iv) relieving the disease, that is, causing the regression of clinical symptoms by administration of a protective composition after their initial appearance.

### 1. Prevention of Cancer

Disclosed herein, in some embodiments, are methods for preventing development of cancer (i.e. carcinogenesis) comprising providing to a subject in need thereof a TfR1-binding protein, for example a TfR1-binding protein disclosed herein. The cancer may be a solid tumor, metastatic cancer, non-metastatic cancer, or hematopoetic cancer. In certain embodiments, the cancer may originate in the bone marrow, bone, cartilage, brain, breast, bladder, kidney, ureter, uterus-endometrial, cervix-endocervix, esophagus, stomach, duodenum, small intestine, appendix, cecum, colon, rectum, anal canal, head and neck, salivary glands, thyroid, pancreatobilliary, spleen, liver, lung, oropharynx, larynx, ovary, fallopian tubes, prostate, testis, eye, skin, adipose tissue, synovium, nerve cell/sheath, or thymus.

The cancer may specifically be of one or more of the following tissue origin: glandular epithelium, superface epithelium, fibroblasts, cartilage/bone, striate muscle, smooth muscle, blood vessels, endothelium, fat, neuroectoderm, hepatocytes, and chorionic epithelium. There are different histological types of malignancies (non-epithelial tumors and epithelial tumors). The cancer may specifically be of one or more of the following histological types, though it is not limited to these: liposarcoma, fibrosarcoma, myxosarcoma, chondrosarcoma, osteosarcoma, synovial sarcoma, epithelioid sarcoma, epithelioid angiosarcoma, alveolar soft part sarcoma, malignant fibrous histiocytoma, leiomyosarcoma, rhabdomyosaroma, embryonal rhabdomyosarcoma, alveolar rhabdomyosarcoma, cystosarcoma phyllodes, angiosarcoma, lymphangiosarcoma, invasive meningioma, leukemias, Hodgkin lymphoma (HL) and non-Hodgkin lymphoma (NHL), multiple myeloma (MM) including plasma cell leukemia, mast cell leukemia/sarcoma, erytroleukemia, myeloid leukemia/sarcoma, basophilic leukemia, eosinophilic leukemia, monocytic leukemia, hairy cell leukemia, neurogenic sarcoma, Kaposi's sarcoma, granular cell tumor, gastrointestinal stromal tumor, neuroblastoma, medulloblastoma, retinoblastoma, melanoma including amelanotic, malignant teratomas, primitive neuroectodermal tumor, Ewing's sarcoma, glioblastoma, astrocytoma, neurofibrosarcoma, adamantinoma, chordoma, ependymoma, astrocytoma, oligoendroblastoma, cerebelar sarcoma, germ-cell tumors of ovary and testes (seminoma, dysgerminoma, gynandroblastoma), non-germ cell tumors (embryonic carcinoma, choriocarcinoma, yolk sac tumor, immature teratoma, teratocarcinoma, sex chord-stromal tumors (granulosa cell and Sertoli-Leydig cell tumors). Malignancies also include undifferentiated carcinoma, well-differentiated carcinoma, keratinizing and nonkeratinizing squamous cell carcinoma, basaloid squamous cell carcinoma, NUT midline carcinoma, spindle cell carcinoma, giant cell carcinoma, pleomorphic carcinoma, transitional cell carcinoma, adenocarcinoma, lepidic adenocarcinoma, acinar adenocarcinoma, papillary adenocarcinoma, solid adenocarcinoma, micropapillary adenocarcinoma, mucinous adenocarcinoma, epithelial myoepithelial carcinoma, adenosquamous carcinoma, basal cell carcinoma, large cell carcinoma, large cell neuroendocrine carcinoma, mucoepidermoid carcinoma, adenoid cystic carcinoma, acinic cell carcinoma, hepatocellular carcinoma, cholangiocarcinoma, choriocarcinoma, neuroendocrine carcinoma, lymphoepithelial carcinoma, thymoma, thymic carcinoma, thyroid carcinoma (anaplastic, hurthle cell, papillary, follicular, and medullary thyroid carcinoma), cutaneous squamous cell carcinoma, Paget's disease of the anus, clear cell renal cell carcinoma, cervical carcinoma, urothelial carcinoma, small and non-mall cell carcinoma of lung, endometrial adenocarcinoma, adrenocortical carcinoma, chromophobe renal cell carcinoma, granular cell carcinoma, malignant mesothelioma, skin appendages carcinoma (hair, nails, sebaceous glands, sweat glands and mammary glands), Merkel cell carcinoma, pilomatrix carcinoma, apocrine gland carcinoma, papillary eccrine carcinoma, sebaceous adenocarcinoma, mucoepidermoid carcinoma, invasive and noninvasive ductal/lobular carcinoma of breast, inflammatory breast carcinoma, Paget's disease of the breast/nipple and areola, medullary carcinoma, colloid (mucinous) carcinoma including signet ring variant, papillary carcinoma, tubular carcinoma, adenoid cytic carcinoma, secretory carcinoma, carcinoma with metaplasia, ovarian surface epithelium-stroma tumors including serous, mucinous, endometrioid, clear cell, Brunner cells, and transitional cells tumors.

In some embodiments, the cancer is a cancer caused by and/or associated with an infectious agent, such as a virus. Examples of infectious agents that are associated with cancers (also “cancer-associated infectious agents”) include hepatitis B virus (HBV), hepatitis C virus (HCV), Epstein-Barr virus (EBV), human papillomavirus (HPV), human immunodeficiency virus (HIV), human T-cell leukemia/lymphoma virus type 1 (HTLV-1), Kaposi sarcoma-associated herpesvirus (KSHV), and Merkel cell polyomavirus (MCPyV). In some embodiments, the infectious agent is EBV. In some embodiments, the infectious agent is HIV. Examples of cancers to be treated or prevented that may be caused by or associated with an infectious disease (also “infectious agent-associated cancers”) include hepatocellular carcinoma, Burkitt lymphoma, nasopharyngeal carcinoma, cervical cancer, Hodgkin lymphoma (HL), non-Hodgkin lymphoma (NHL), Kaposi sarcoma, adenocarcinoma, and adult T-cell leukemia/lymphoma (ATL). In some embodiments, the cancer is an EBV-associated lymphoma. In some embodiments, the cancer is an EBV-associated HL. In some embodiments, the cancer is an EBV-associated NHL. In some embodiments, the cancer is an HIV-associated B-cell lymphoma. Aspects of the disclosure include detecting an infectious agent in an individual before, during, and/or after treatment. Detecting an infectious agent may be excluded from embodiments of the present disclosure.

Methods may involve the determination, administration, or selection of an appropriate cancer “management regimen” and predicting the outcome of the same. As used herein the phrase “management regimen” refers to a management plan that specifies the type of examination, screening, diagnosis, surveillance, care, and treatment (such as dosage, schedule and/or duration of a treatment) provided to a subject in need thereof (e.g., a subject at risk of developing cancer).

The selected treatment regimen can be an aggressive one which is expected to result in the best clinical outcome (e.g., complete prevention or cure of the disease) or a more moderate one which may relieve symptoms of the disease yet results in incomplete cure or prevention of the disease. The type of treatment can include a surgical intervention, administration of a therapeutic drug such as a TfR1-binding protein, immunotherapy, an exposure to radiation therapy and/or any combination thereof. The dosage, schedule and duration of treatment can vary, depending on the severity of disease and the selected type of treatment, and those of skill in the art are capable of adjusting the type of treatment with the dosage, schedule and duration of treatment.

Biomarkers like TfR1 that can predict the efficacy of certain therapeutic regimen and can be used to identify patients who will receive benefit of a conventional single or combined modality therapy before treatment begins or to modify or design a future treatment plan after treatment. In the same way, those patients who do not receive much benefit from such conventional single or combined modality therapy and can offer them alternative treatment(s) may be identified.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. The Examples should not be construed as limiting in any way. The contents of all cited references (including literature references, issued patents, published patent applications, and GenBank Accession numbers as cited throughout this application) are hereby expressly incorporated by reference. When definitions of terms in documents that are incorporated by reference herein conflict with those used herein, the definitions used herein govern.

### Example 1—In Vivo Efficacy of Anti-TfR1 Antibody in Prevention of EBV-Induced Carcinogenesis

In vivo efficacy of ch128.1/IgG1 antibodies in preventing EBV-induced carcinogenesis was evaluated.

### Methods

### Isolation of Human B-Cell Enriched Preparations

Peripheral blood mononuclear cells (PBMCs), isolated from healthy anonymous donors, were obtained from the UCLA AIDS Institute Virology Core laboratory. B-cell enriched preparations were obtained by incubating PBMCs with CD3 superparamagnetic beads coupled with anti-human CD3 antibody (Dynal Biotech, Oslo, Norway) to deplete human CD3⁺ T-cells.

### Infection of Human B-Cells by EBV

Human B-cell encirhced preparations were infected with EBV by exposing cells to supernatants from B-95-8 EBV-infected marmoset cells, which contain a high concentration of infectious EBV. After exposure to EBV-containing supernatants, the B-cell enriched cells were washed two times with phosphate buffered saline (PBS). EBV-exposed B-cell preparations were placed in culture for 1 week, then implanted into animals (Groups 3 and 4).

### Preparation of Antibodies

The IgG1 version of the ch128.1 antibody (ch128.1/IgG1) (Daniels-Wells et al., J. Immunother., 43(2):48-52 (2020)) was tested. A mouse/human chimeric IgG1 antibody specific for the hapten dansyl (5-dimethylamino naphthalene-1-sulfonyl chloride)) (Tao et al., J. Exp. Med., 173(4):1025-1028 (1991)) was used as an isotype (IgG1) control. Both antibodies were expressed in murine myeloma cells, which were grown in roller bottles and the antibodies purified using affinity chromatography.

### Inoculation into Immunodeficient Mice

Immunodeficient NOD.Cg-Prkdc^{scid}Il2rg^{tm1Wjl}/SzJ (NSG) mice were used for these studies. These mice are highly immunodeficient; they carry two mutations on the NOD/ShiLtJ genetic background, severe combined immune deficiency (scid) and a complete null allele of the IL2 receptor common gamma chain (IL2rg^{null}). The scid mutation is in the DNA repair complex protein Prkdc and renders the mice B and T-cell deficient, as they cannot produce functional T or B-cell antigen receptors. The IL2rg^{null} mutation prevents cytokine signaling through multiple receptors, leading to a deficiency in functional NK cells. There were six groups, as described below:
Group 1: Mice implanted with human B-cell enriched preparations not infected with EBV and treated with isotype control IgG1.
Group 2: Mice implanted with human B-cell enriched preparations not infected with EBV and treated with ch128.1/IgG1.
Group 3: Mice implanted with EBV-exposed B-cell enriched preparations (cultured in vitro for 7 days) and treated with isotype control IgG1.
Group 4: Mice implanted with EBV-exposed B-cell enriched preparations (cultured in vitro for 7 days) and treated with ch128.1/IgG1.

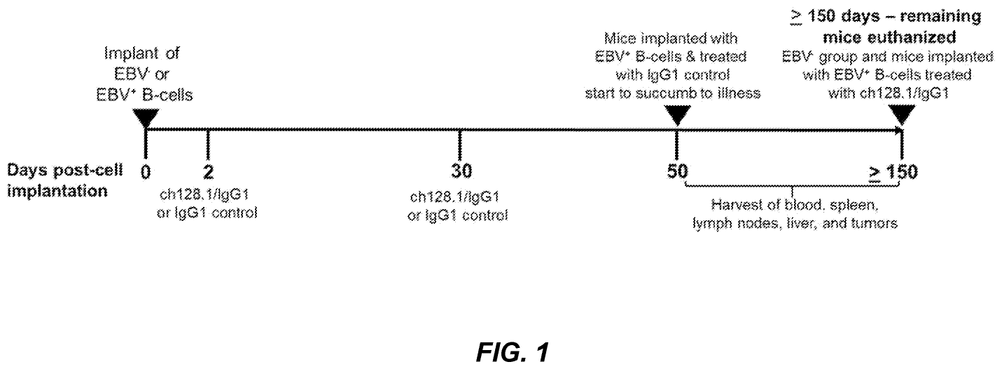

Groups 1 and 2 had 13 mice each while groups 3 and 4 had 14 mice each. Human primary B-cells were infected with EBV by exposing T-cell depleted, B-cell enriched PBMC preparations to supernatants from B95-8 EBV-infected marmoset cells, which contain a high concentration of infectious EBV, as described (Martinez-Maza and Britton, J. Exp. Med., 157:1808-476 (1983)). T-cell depleted PBMCs were exposed to EBV supernatants or media alone for two hours in vitro and then washed with PBS. EBV-exposed cells were then cultured in vitro for 7 days, after which these preparations were washed twice with PBS. Mice were implanted intravenously (i.v.) with human B-cell enriched cell preparations (6×10⁶ cells in 0.25 ml PBD per mouse) at day 0, and then administered i.v. via the tail vein with 400 μg of ch128.1/IgG1 or with isotype control IgG1, 2 days after that, and again 28 days later. Mice were euthanized when they showed overt signs of tumor growth or physical distress, or in the absence of that, at or after 150 days post-cell implantation as shown in FIG. 1. Animals were monitored for tumor development and euthanized when moribund or when they showed overt signs of illness or physical distress. Survival was recorded as the number of days from cell implantation to euthanasia. At necropsy, blood was collected via cardiac puncture and spleen, lymph node, and liver tissues were collected. Tissues from mice that developed tumor-like growths were also obtained at necropsy. The remaining, surviving mice that did not show signs of malaise or tumor development were euthanized at or after 150 days post-cell implantation with EBV⁻ or EBV⁺ B-cells.

### Flow Cytometry Studies

Multicolor flow cytometry was carried out to determine the presence of human immune cells in the mice, as previously described (Epeldegui, et al., Sci Rep 9(1): Article 9371 (2019)). Briefly, cells were isolated from tissues by mechanical dissociation using a rubber syringe tip to grind up tissue and liberate cells. Cells were then suspended in red blood cell (RBC) lysis buffer, passed through 70 μm cell strainers (Corning Inc, Corning, N.Y.), and incubated for 5 minutes at room temperature to lyse RBCs. Cells were then washed once with fluorescence-activated cell sorting (FACS) buffer (0.5% bovine serum albumin (BSA) in PBS) and then 5 μl of Super Bright Complete Staining Buffer (eBioscience, San Diego, Calif.) was added to each sample, prior to introducing the antibody cocktail mix, which is used when more than one Super Bright polymer-dye conjugated antibody is added to the same sample to prevent non-specific polymer interactions. An antibody cocktail was then added consisting of: anti-human CD3 (hCD3) monoclonal antibody (OKT3, Super Bright 600, eBioscience), anti-human CD45 (hCD45) monoclonal antibody (HI30, eFluor 450, eBioscience), anti-human CD19 (hCD19) monoclonal antibody (SJ25C1, PerCP-Cyanine5.5, eBioscience), and anti-murine CD45 (mCD45) monoclonal antibody (30-F11, Super Bright 645, eBioscience). Cells were incubated with the antibody cocktail for 20 minutes at 4° C., washed once in FACS buffer, and centrifuged at 1,500 rpm for 5 minutes. After the centrifugation, the solution was discarded and the cell pellet was resuspended in approximately 0.25 ml of cold 1% paraformaldehyde solution in PBS. Cells were analyzed by flow cytometry using a BD LSRFortessa™ X-20 Cell Analyzer (BD Biosciences), and data were analyzed with the FCS Express software program (v7.0, De Novo Software, Pasadena, Calif.).

### Immunohistochemical Studies

Tissues from mice that developed tumor-like growths were obtained at necropsy, fixed in 10% neutral buffered formalin for 7 days, and then placed in 70% ethanol for 1-3 days. Immunohistochemical staining was performed at the UCLA Translational Pathology Core Laboratory (TPCL) using the following antibodies: polyclonal rabbit anti-human κ LC (Agilent, Santa Clara, Calif.), polyclonal rabbit anti-human λ LC (Agilent), monoclonal mouse anti-EBV LMP (Clone CS.1-4, Agilent), polyclonal rabbit anti-human CD3 (Agilent), and monoclonal mouse anti-human CD19 (Clone LE-CD19, Agilent). An anti-rabbit horseradish peroxidase (HRP)-labeled polymer conjugated secondary antibody (EnVision+/HRP polymer, Rabbit) (Agilent) was used to recognize rabbit anti-human primary antibodies. An anti-mouse, HRP-labeled polymer conjugated secondary antibody (EnVision+/HRP polymer, Mouse) (Agilent) was used to recognize mouse anti-human primary antibodies. For detection of rabbit or mouse specific antibodies, a 3,3′-Diaminobenzidine (DAB) chromogen and DAB substrate buffer kit was used (DAB Chromogen Kit) (Biocare Medical, Pacheco, Calif.). These tissues were then examined for features characteristic of lymphoma-like tumors, including the presence of B-cells (CD19 positivity), evidence of EBV-infection (LMP1-staining), and evidence for monoclonality (staining for either κ or λ immunoglobulin light chain). Lymphoma-like tumors would be expected to be of B-cell origin, monoclonal (either κ or λ positive, but not positive for both), and to show evidence for EBV infection (e.g., LMP1 positivity).

### Immunometric Assays and Enzyme-Linked Immunosorbent Assays (ELISA)

Multiplexed immunometric assays (Luminex, Austin, Tex.) and ELISA were carried out to quantify plasma levels of human cytokines, soluble receptors, and immunoglobulins. Multiplexed-immunometric assays were carried out using a custom made panel (R&D Systems, Minneapolis, Minn.) to quantify plasma levels of markers associated with B-cell activation and/or survival, such as key human pro-inflammatory (IL-6, IFN-γ, and TNF-α,) and anti-inflammatory (IL-10) cytokines, pro-inflammatory chemokines (IL-8 and CXCL10), and soluble receptors (sCD25 and sCD27). A separate multiplex panel was used for the simultaneous quantification of human immunoglobulins (IgA, IgG1, IgG2, IgG3, IgG4, and IgM) (Bio-Rad, Hercules, Calif.). Multiplexed assays were quantified using a BioPlex 200 (Luminex) System Analyzer (Bio-Rad, Hercules, Calif.), and the data were analyzed using BioPlex Manager (v 4.1.1) software (Bio-Rad). The lower limit of detection for each biomarker was set either as the lowest value that the BioPlex Manager software could calculate using the standard curve, or as the lowest value of the standard curve, whichever was smaller. For quality control, samples were equally distributed across reaction plates, and replicates were included across the reaction plates to calculate coefficients of variation. Plasma levels of human immunoglobulin κ or λ free light chain (FLC) were assessed using an ELISA kit (BioVendor, Brno, Czech Republic) according to the manufacturer's instructions.

### Results

### Mice Implanted with EBV-Infected Human B-Cells Die in the Absence of Treatment with ch128.1/IgG1

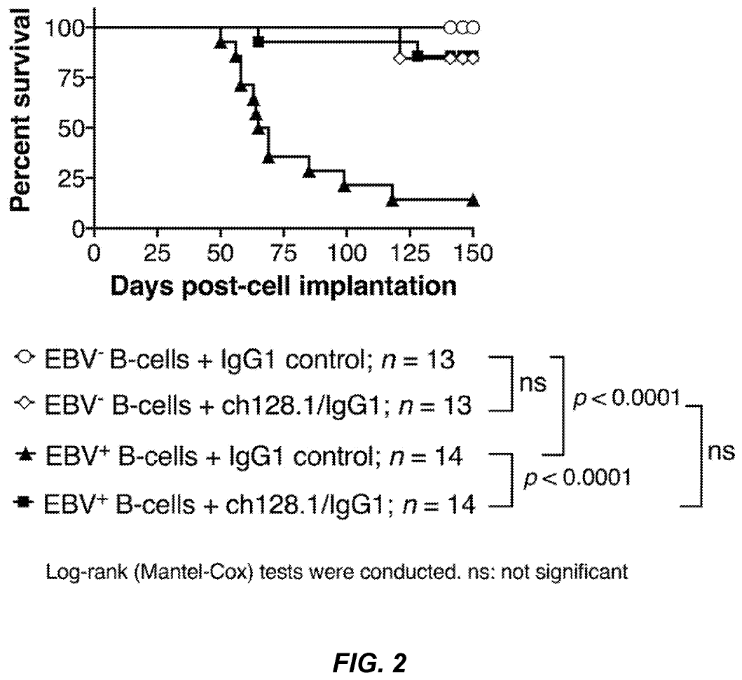

Most (12/14) mice implanted with EBV-exposed human B-cell enriched preparations and injected with isotype control IgG1 (Group 3), were seen to die by 150 days post-cell implantation. In contrast, all but 2 mice that were treated with ch128.1/IgG1 and implanted with EBV-exposed cells (Group 4) survived past 150 days, as shown in FIG. 2. The difference in survival between the ch128.1/IgG1-treated group (Group 4) and the isotype control group (Group 3), both of which were implanted with EBV-exposed cells, was statistically significant (FIG. 2); p≤0.05 were considered statistically significant. There was also no significant difference in survival between the mice that were implanted with cells that were not infected with EBV (Groups 1 and 2) and the ch128.1/IgG1-treated group (Group 4) implanted with EBV-exposed cells (FIG. 2).

Thus, treatment of NSG mice implanted with EBV-infected human B-cell preparations with ch128.1/IgG1 (Group 4), but not with the isotype control IgG1 (Group 3), resulted in a marked enhancement of survival in these mice implanted with EBV-infected human B-cells.

### Mice Implanted with EBV-Infected Human Cells Developed Monoclonal Growths that were of Human B-Cell Origin and Were EBV⁺

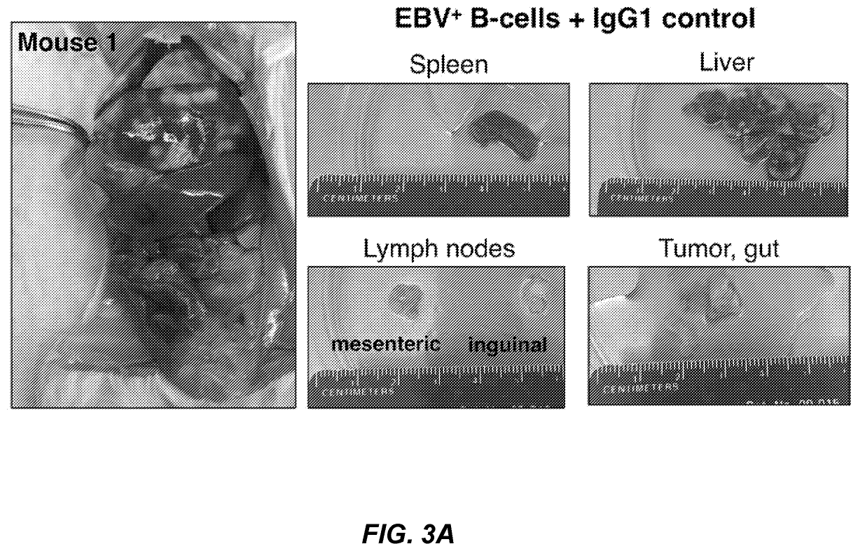

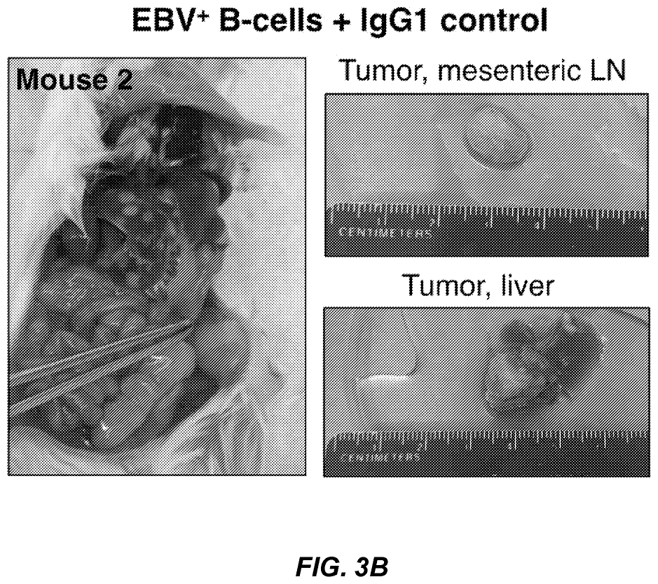

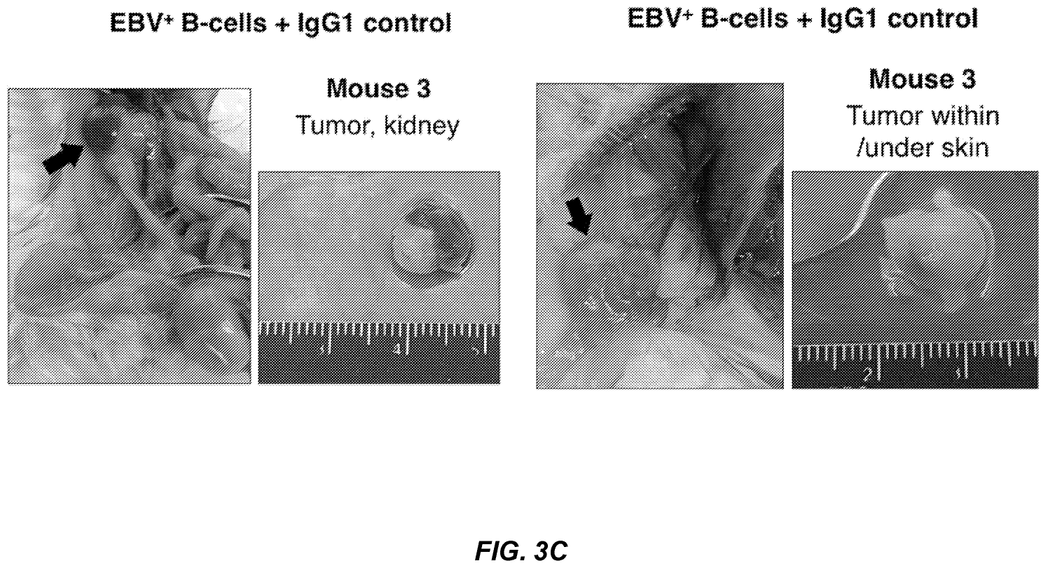

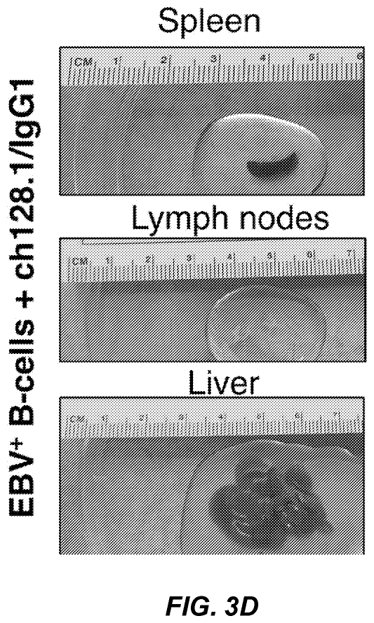

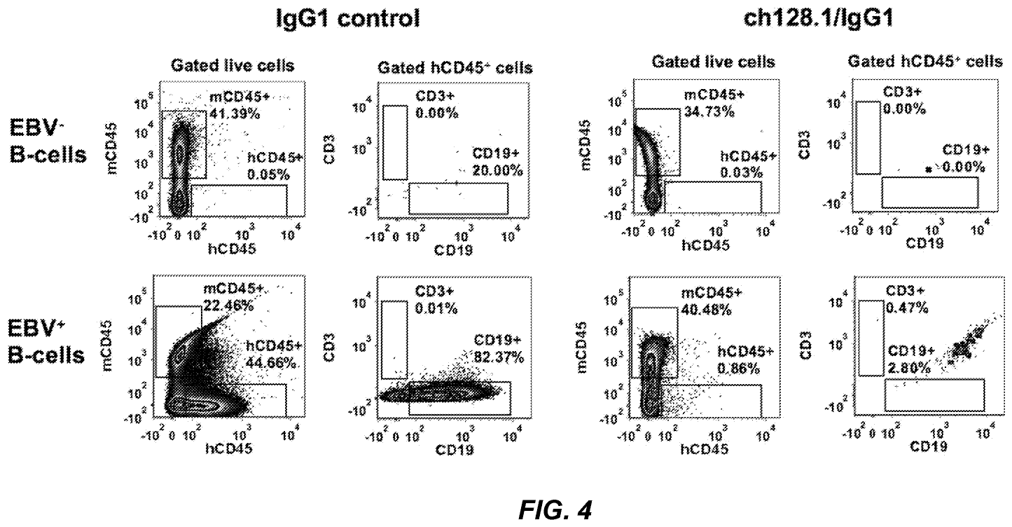

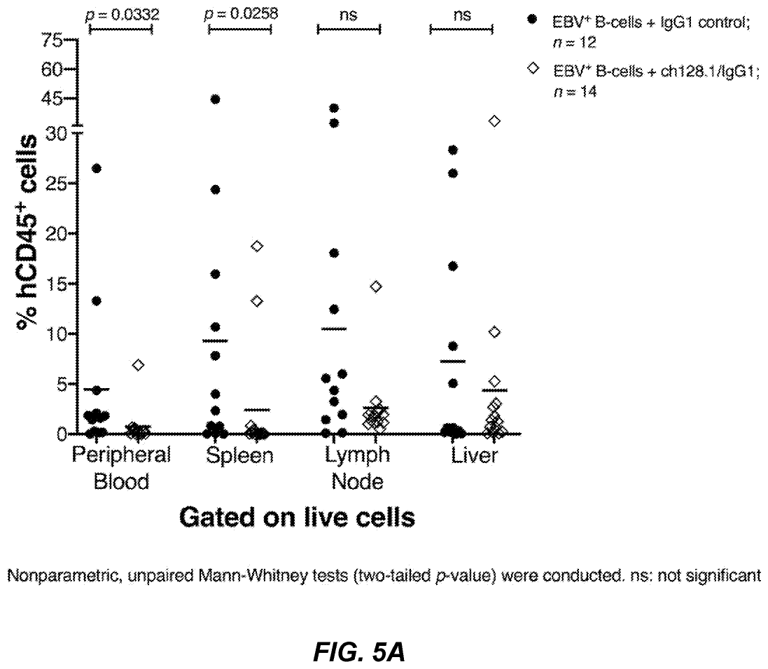

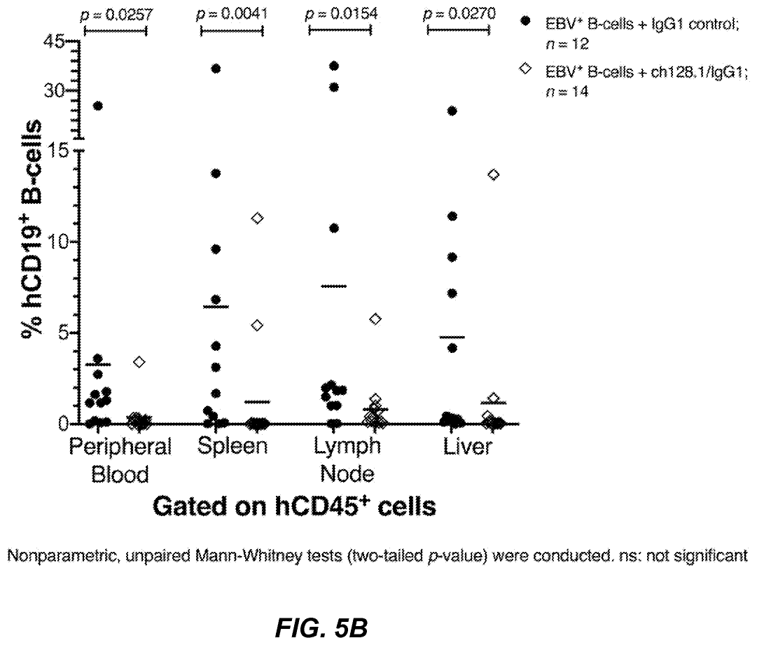

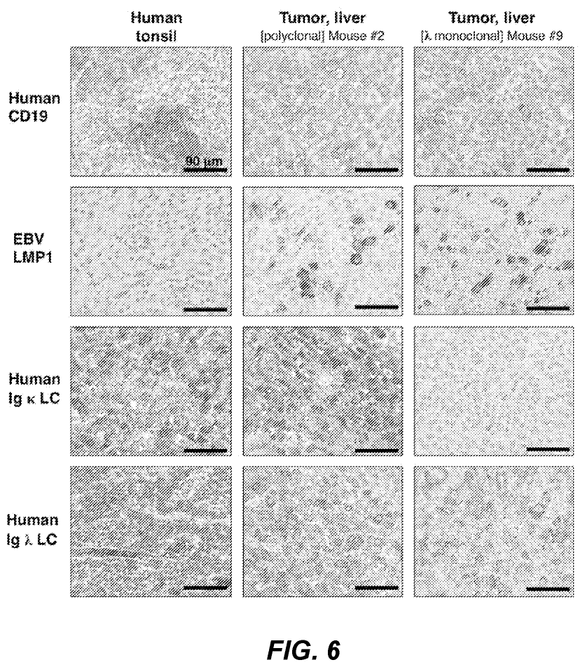

Necropsies were carried out whenever possible in the mice that died, or were sacrificed, prior to the end of the study. Blood and tissues were collected for analysis, including pathology and assessment by flow cytometry. All but one of the mice that died and were necropsied were in the isotype control group (Group 3); one mouse was in the EBV-exposed ch128.1/IgG1 treated group (Group 4). On visual, macroscopic assessment, all of these mice showed diffuse white spots in spleen, liver, lymph nodes, kidneys, and in some cases, tumors near the gastrointestinal tract or under the skin (FIGS. 3A-3C). In contrast, most of the 14 mice treated with ch128.1/IgG1 showed normal tissues without lymphoproliferative growths (FIG. 3D); only 2 of the 14 mice treated with ch128.1/IgG1 showed disseminated disease and developed tumors in the liver (not shown). Flow cytometry analysis showed a dramatic increase in the percentage of hCD45⁺ (human origin) leukocytes that were also CD19⁺ in the spleen of mice that were implanted with EBV⁺ B-cells and treated with the IgG1 control, confirming the growth of B-cells of human origin in these mice (FIG. 4). However, cells analyzed from various tissues (peripheral blood, spleen, lymph node, and liver) contained little (<1%) to no detectable human CD3⁺ T-cells (spleen (FIG. 4); other tissues (not shown)). Most mice implanted with EBV⁺ B-cells and treated with ch128.1/IgG1 (Group 4) did not have detectable hCD45⁺ cells (cells or human origin) in blood, lymph node, spleen, and liver tissues (FIGS. 5A-5B). Only 2 mice in that group showed hCD45⁺ cells out of the total 14 mice treated with ch128.1/IgG1. In contrast, mice in the IgG1 control monoclonal antibody (Group 3) had hCD45⁺ (human) and hCD19⁺ (B-cells) cells in blood, lymph node, spleen, and liver tissues (FIGS. 5A-5B). However, it was not possible to harvest blood or collect any tissues from 2 of the mice in the isotype control group (Group 3), so FIGS. 5A-5B show results for only 12 of the 14 animals in that group. Mice implanted with EBV-exposed cells and treated with IgG1 control monoclonal antibody (Group 3) developed lymphoma-like growths that were of human B-cell origin (CD19⁺) and EBV LMP1-positive, by immunohistochemical analysis (areas that are darker indicate positive staining) (FIG. 6). Only in a few cases were human CD3⁺ T-cells detected in tumor tissues, which were mainly composed of human CD19⁺ B-cells (not shown). In order to assess the clonality of tumors, the expression of human κ or λ light chain (LC) was determined. Although most tumor tissues showed both positive staining for human κ and λ LC (such as is seen in FIG. 6, middle panel Mouse #2), some had exclusively κ or λ LC positivity (such as is seen in FIG. 6, right panel Mouse #9, which is negative for κ LC but positive for λ LC), suggesting that these were monoclonal B-cell proliferations. No cancer-like lesions were seen in the control groups that were implanted with human B-cells not infected with EBV (Groups 1 and 2). It was notable that only 2 animals showing these lymphoma-like lesions were in the ch128.1/IgG1-treated groups, with most of the animals displaying these cancer-like lesions in the isotype control groups. Therefore, human B-cell lymphoma like cells (i.e., human B-cells that were EBV⁺ and showed evidence for monoclonality) were clearly seen in several animals implanted with EBV-exposed human B-cells, mainly in those not treated with ch128.1/IgG1.

### Treatment with ch128.1/IgG1 Significantly Reduced Plasma Levels of Markers of Inflammation and B-Cell Activation in Mice Implanted with EBV⁺ B-Cells

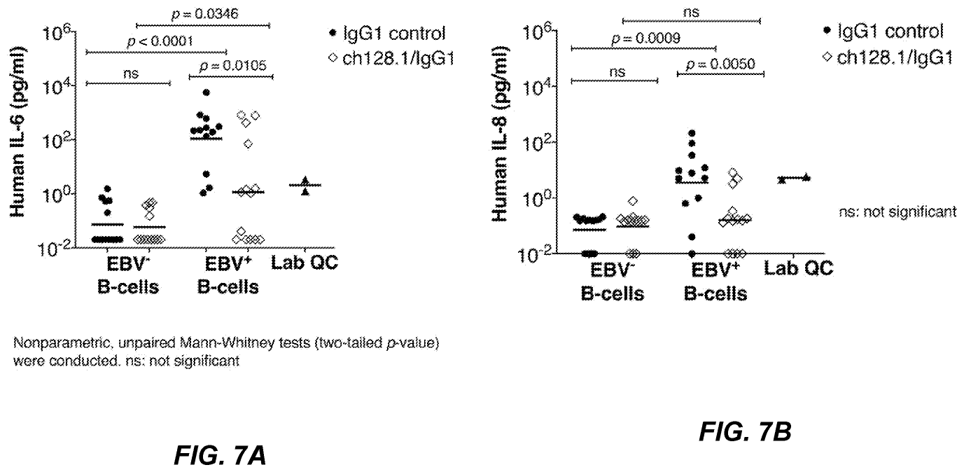

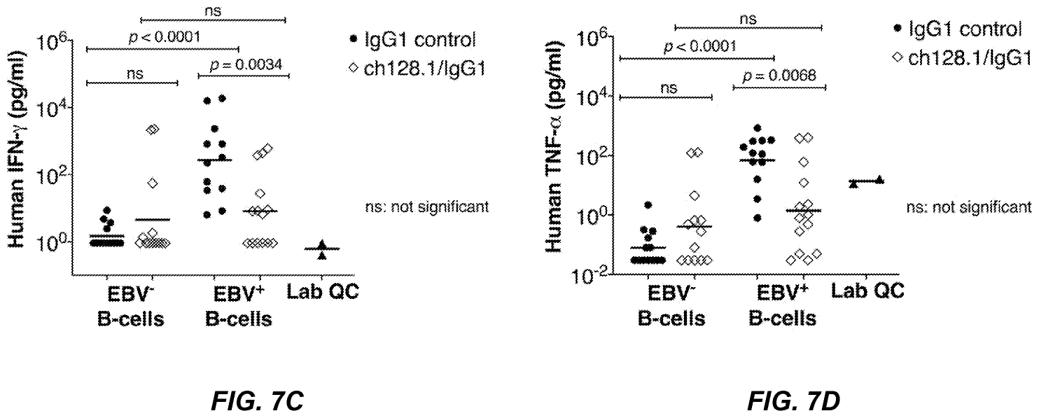

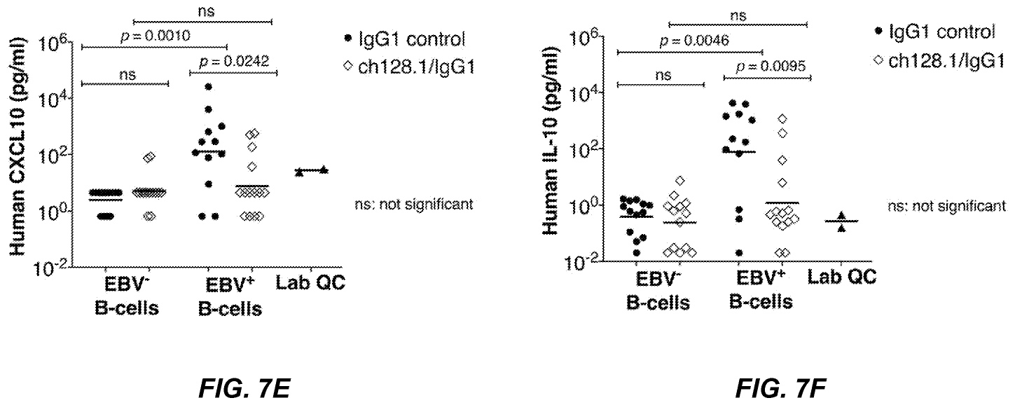

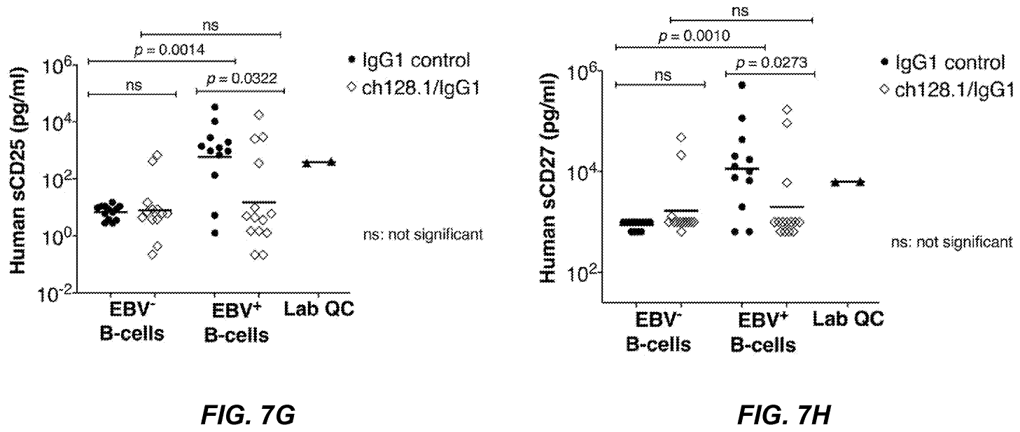

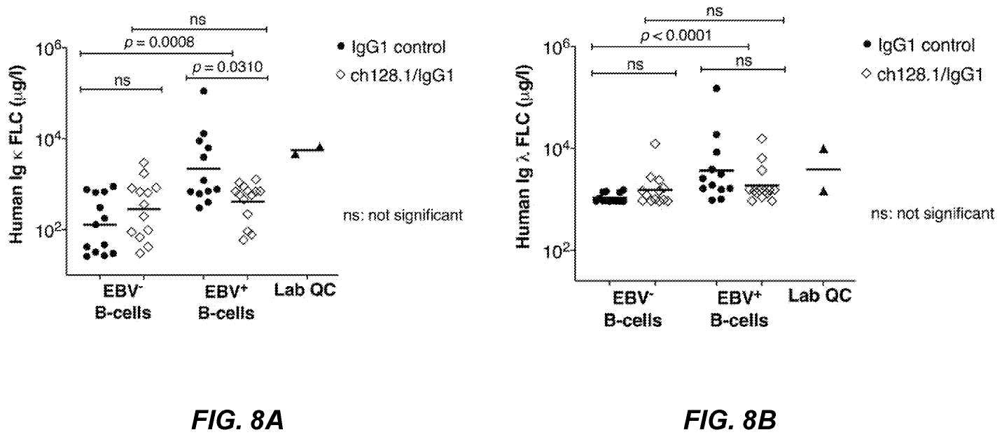

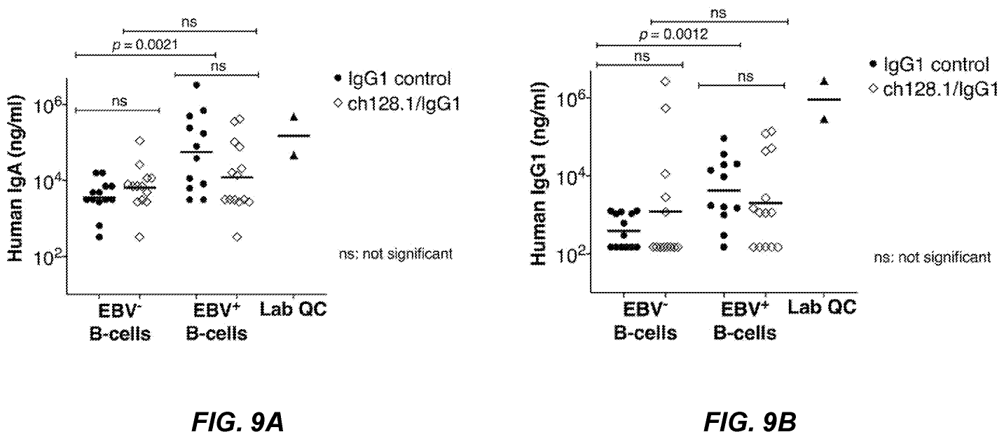

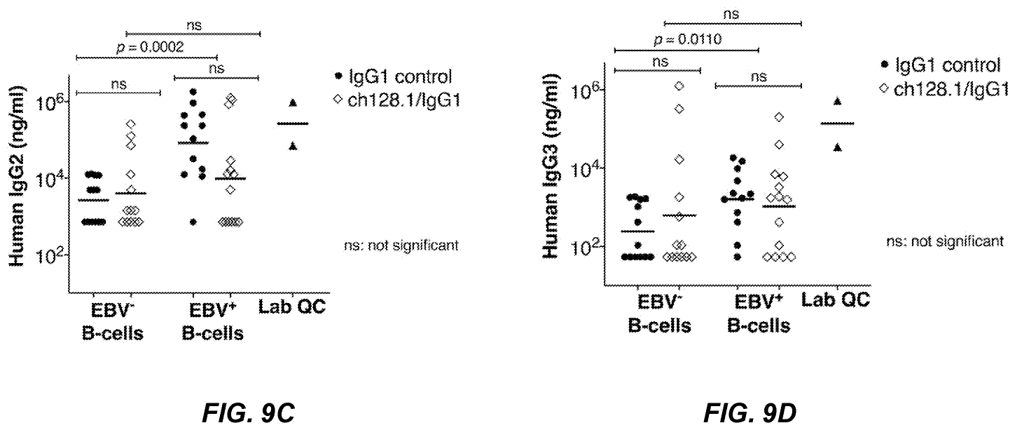

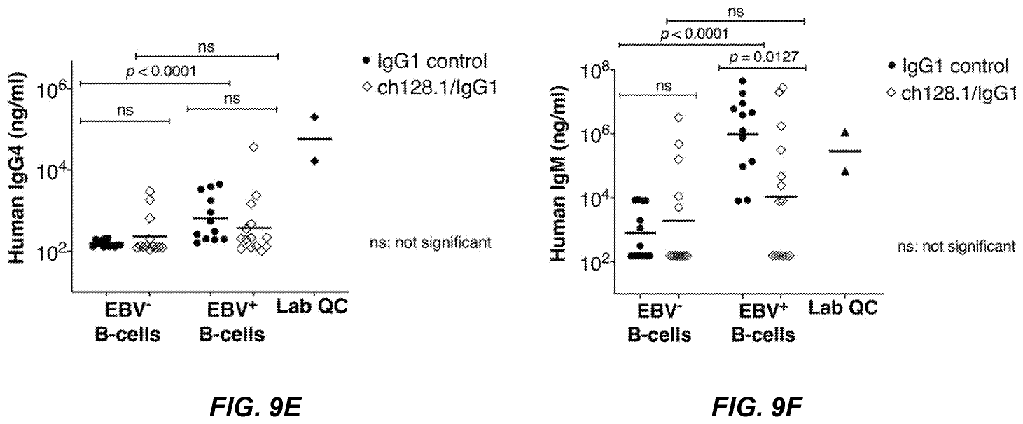

Plasma collected from all available animals at death was used to quantify in vivo levels of human cytokines and chemokines (IL-6, IL-8, IL-10, TNF-α, IFN-γ, and CXCL10), soluble receptors (sCD25 and sCD27) and immunoglobulins (IgA, IgM, IgG1, IgG2, IgG3, IgG4), by multiplexed immunometric assay (Luminex), as well as human κ or λ LC by ELISA. Laboratory quality control (Lab QC) specimens (plasma from healthy control donors) were included as additional assay controls for these studies in FIGS. 7A-H, FIGS. 8A-B, and FIGS. 9A-F. Statistical significance in those figures was determined using the nonparametric, unpaired Mann-Whitney test (two-tailed p-value), as noted in FIG. 7A. As expected, infection with EBV increased the levels of inflammatory cytokines. This increase was diminished by treatment with ch128.1/IgG1, which is consistent with inhibition of lymphomagenesis. Treatment with ch128.1/IgG1 resulted in significantly reduced plasma levels of human pro-inflammatory molecules IL-6, IL-8, IFNγ, TNFα, and CXCL10 (FIGS. 7A-E). In addition, reduced levels of IL-10, sCD25, and sCD27 were observed in the ch128.1/IgG1 treated mice (FIGS. 7F-H). It was found that plasma κ and λ FLC concentrations were significantly elevated in mice implanted with EBV⁺ B-cells and treated with IgG1 control (Group 3) compared to its respective control group, EBV⁻ B-cell implanted mice treated with IgG1 control (Group 1), suggesting increased B-cell activation in mice implanted with EBV⁺ B-cells (FIGS. 8A-8B). Furthermore, mice implanted with EBV⁺ cells and treated with ch128.1/IgG1 showed significantly reduced levels of κ FLC compared to EBV⁺ mice treated with IgG1 control (FIG. 8A); λ FLC levels were also decreased in mice treated with ch128.1/IgG1 compared to EBV-infected mice treated with IgG1 control, although this was not statistically significant (FIG. 8B). Moreover, we found that there was a correlation between κ or λ LC positivity in mice that developed monoclonal tumor-growths and their respective plasma Ig κ and λ FLC concentrations (not shown). Mice implanted with EBV⁺ B-cells had elevated plasma levels of all classes of immunoglobulin molecules, compared to mice implanted with EBV⁻ B-cells (FIGS. 9A-9F). Reduced plasma levels of human IgA and IgG1, IgG2, IgG3 or IgG4 were observed for mice implanted with EBV⁺ B-cells and treated with ch128.1/IgG1 versus those treated with IgG1 control antibody, although this was not statistically significant (FIGS. 9A-9E). IgM levels were significantly lower in mice implanted with EBV⁺ B-cells and treated with ch128.1/IgG1 compared to mice treated with IgG1 control (FIG. 9F).

### CONCLUSIONS

Implantation of immunodeficient mice with EBV-exposed human B-cells resulted in the development of B-cell lymphoma-like cancers, and in death within 150 days post-cell implantation in most animals. The lymphoma-like growths that were seen in the spleens, livers, and other tissues of these animals contained EBV⁺ human B-cells, which were monoclonal in some instances. Therefore, this is a meaningful animal model for the transformation and immortalization of EBV-infected human B-cells, and for their development into lymphoma-like tumors.

Treatment of mice implanted with EBV-exposed human B-cells with ch128.1/IgG1, but not with isotype control IgG1, was seen to inhibit the growth of EBV⁺ human B-cells in vivo, as well as the development of these cells into lymphoma-like lesions. Similarly, treatment with ch128.1/IgG1 or anti-TfR1 resulted in decreased plasma levels of IL-6 and other cytokines in these mice. IL-6 is a B-cell stimulatory cytokine that is associated with the development of lymphoma and other lymphopoietic tumors.

Overall, the anti-TfR1 antibody ch128.1/IgG1 proved to be an an effective agent for the inhibition of the growth of EBV⁺ B-cells in vivo, as well as the development of these cells into lymphoma-like tumors. Thus, there is a clear role for ch128.1/IgG1 treatment in the prevention of B-cell malignancies.

All of the methods disclosed and claimed herein can be made and executed without undue experimentation in light of the present disclosure. While the compositions and methods of this disclosure have been described in terms of certain embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

The references recited in the application, to the extent that they provide exemplary procedural or other details supplementary to those set forth herein, are specifically incorporated herein by reference.

What is claimed is:

1. A method for preventing cancer comprising:
(a) detecting the presence of an infectious agent in a subject; and
(b) providing to the subject an effective amount of a transferrin receptor 1(TfR1)-binding protein.

2. The method of claim 1, wherein the TfR1-binding protein is an anti-TfR1 antibody or a fragment thereof.

3. The method of claim 2, wherein the TfR1-binding protein is a mouse anti-TfR1 antibody or a fragment thereof.

4. The method of claim 2, wherein the TfR1-binding protein is a chimeric anti-TfR1 antibody or a fragment thereof.

5. The method of claim 4, wherein the TfR1-binding protein is a ch128.1 antibody.

6. The method of claim 5, wherein the TfR1-binding protein is ch128.1/IgG1.

7. The method of claim 5, wherein the TfR1-binding protein is ch128.1/IgG3.

8. The method of claim 2, wherein the TfR1-binding protein is a human or humanized anti-TfR1 antibody or a fragment thereof.

9. The method of any of claims 1-8, wherein the TfR1-binding protein is an aptamer capable of binding to TfR1.

10. The method of any of claims 1-9, wherein the infectious agent is a cancer-associated infectious agent.

11. The method of any of claims 1-10, wherein the infectious agent is hepatitis B virus (HBV), hepatitis C virus (HCV), Epstein-Barr virus (EBV), human papillomavirus (HPV), human immunodeficiency virus (HIV), human T-cell leukemia/lymphoma virus type 1 (HTLV-1), Kaposi sarcoma-associated herpesvirus (KSHV), or Merkel cell polyomavirus (MCPyV).

12. The method of claim 11, wherein the infectious agent is EBV.

13. The method of claim 11, wherein the infectious agent is HIV.

14. The method of any of claims 1-13, wherein the cancer is non-Hodgkin lymphoma (NHL), Hodgkin lymphoma (HL), Burkitt lymphoma, primary effusion lymphoma (PEL), B-cell lymphoma of the elderly, adult T-cell leukemia/lymphoma (ATL), Kaposi sarcoma, hepatocellular carcinoma, cervical cancer, gastric cancer, or nasopharyngeal carcinoma.

15. The method of claim 14, wherein the cancer is NHL.

16. The method of claim 14, wherein the cancer is HL.

17. The method of any of claims 1-16, wherein the subject has or is at risk for developing post-transplant lymphoproliferative disorder (PTLD).

18. The method of any of claims 1-17, further comprising providing an additional therapeutic.

19. The method of claim 18, wherein the additional therapeutic is covalently attached to the TfR1-binding protein.

20. The method of claim 18, wherein the additional therapeutic is non-covalently attached to the TfR1-binding protein.

21. The method of claim 18, wherein the additional therapeutic is not attached to the TfR1-binding protein.

22. The method of claim 21, wherein the TfR1-binding protein and the additional therapeutic are provided to the subject substantially simultaneously.

23. The method of claim 21, wherein the TfR1-binding protein and the additional therapeutic are provided to the subject sequentially.

24. The method of any of claims 18-23, wherein the additional therapeutic is a chemotherapeutic drug, a nucleic acid, a protein, a viral vector, or a nanodrug.

25. The method of claim 24, wherein the nucleic acid is an antisense oligonucleotide, a small interfering RNA (siRNA), or a clustered regularly interspaced short palindromic repeats (CRISPR)-based gene therapy.

26. The method of claim 24, wherein the protein is a toxin or an enzyme.

27. The method of any of claims 1-26, wherein the method comprises eliminating pre-malignant cells from the subject.

28. The method of any of claims 1-27, wherein the method comprises inhibiting proliferation of pre-malignant cells from the subject.

29. A method for preventing EBV-associated cancer comprising:
(a) detecting the presence of EBV in a subject; and
(b) providing to the subject an effective amount of ch128.1/IgG1.

30. The method of claim 29, wherein the cancer is NHL, HL, Burkitt lymphoma, PEL, B-cell lymphoma of the elderly, hepatocellular carcinoma, gastric cancer, or nasopharyngeal carcinoma.

31. The method of claim 30, wherein the cancer is NHL.

32. The method of claim 30, wherein the cancer is HL.

33. The method of any of claims 29-32, wherein the subject has or is at risk for developing PTLD.

34. The method of any of claims 29-33, further comprising providing an additional therapeutic.

35. The method of claim 34, wherein the additional therapeutic is covalently attached to the TfR1-binding protein.

36. The method of claim 34, wherein the additional therapeutic is non-covalently attached to the TfR1-binding protein.

37. The method of claim 34, wherein the additional therapeutic is not attached to the TfR1-binding protein.

38. The method of claim 37, wherein the TfR1-binding protein and the additional therapeutic are provided to the subject substantially simultaneously.

39. The method of claim 34, wherein the TfR1-binding protein and the additional therapeutic are provided to the subject sequentially.

40. The method of any of claims 34-39, wherein the additional therapeutic is a chemotherapeutic drug, a nucleic acid, a protein, a viral vector, or a nanodrug.

41. The method of claim 40, wherein the nucleic acid is an antisense oligonucleotide, a siRNA, or a CRISPR-based gene therapy.

42. The method of claim 40, wherein the protein is a toxin or an enzyme.

43. A method for preventing cancer comprising providing to a subject an effective amount of a TfR1-binding protein.

44. The method of claim 43, wherein the TfR1-binding protein is an anti-TfR1 antibody or a fragment thereof.

45. The method of claim 44, wherein the TfR1-binding protein is a mouse anti-TfR1 antibody or a fragment thereof.

46. The method of claim 44, wherein the TfR1-binding protein is a chimeric anti-TfR1 antibody or a fragment thereof.

47. The method of claim 46, wherein the TfR1-binding protein is a ch128.1 antibody.

48. The method of claim 47, wherein the TfR1-binding protein is ch128.1/IgG1.

49. The method of claim 47, wherein the TfR1-binding protein is ch128.1/IgG3.

50. The method of claim 44, wherein the TfR1-binding protein is a human or humanized anti-TfR1 antibody or a fragment thereof.

51. The method of any of claims 43-50, wherein the TfR1-binding protein is an aptamer capable of binding to TfR1.

52. The method of any of claims 43-51, wherein the method further comprises detecting an infectious agent in the subject.

53. The method of claim 52, wherein the infectious agent is detected prior to providing the TfR1-binding protein to the subject.

54. The method of claim 52, wherein the infectious agent is detected subsequent to providing the TfR1-binding protein to the subject.

55. The method of any of claims 52-54, wherein the infectious agent is a cancer-associated infectious agent.

56. The method of any of claims 52-55, wherein the infectious agent is HBV, HCV, EBV, HPV, HIV, HTLV-1, KSHV, or MCPyV.

57. The method of claim 56, wherein the infectious agent is EBV.

58. The method of claim 56, wherein the infectious agent is HIV.

59. The method of any of claims 43-58, wherein the cancer is NHL, HL, Burkitt lymphoma, PEL, B-cell lymphoma of the elderly, adult T-cell leukemia/lymphoma, Kaposi sarcoma, hepatocellular carcinoma, cervical cancer, gastric cancer, or nasopharyngeal carcinoma.

60. The method of claim 59, wherein the cancer is NHL.

61. The method of claim 59, wherein the cancer is HL.

62. The method of any of claims 43-61, wherein the subject has or is at risk for developing PTLD.

63. The method of any of claims 43-62, further comprising providing an additional therapeutic.

64. The method of claim 63, wherein the additional therapeutic is covalently attached to the TfR1-binding protein.

65. The method of claim 63, wherein the additional therapeutic is non-covalently attached to the TfR1-binding protein.

66. The method of claim 63, wherein the additional therapeutic is not attached to the TfR1-binding protein.

67. The method of claim 66, wherein the TfR1-binding protein and the additional therapeutic are provided to the subject substantially simultaneously.

68. The method of claim 66, wherein the TfR1-binding protein and the additional therapeutic are provided to the subject sequentially.

69. The method of any of claims 63-68, wherein the additional therapeutic is a chemotherapeutic drug, a nucleic acid, a protein, a viral vector, or a nanodrug.

70. The method of claim 69, wherein the nucleic acid is an antisense oligonucleotide, a siRNA, or a CRISPR-based gene therapy.

71. The method of claim 69, wherein the protein is a toxin or an enzyme.

72. The method of any of claims 43-71, wherein the method comprises eliminating pre-malignant cells from the subject.

73. The method of any of claims 43-72, wherein the method comprises inhibiting proliferation of pre-malignant cells from the subject.
